(19) United States
(12) Patent Application Publication
(10) Pub. No.: US 2004/0181099 A1
(43) Pub. Date: Sep. 16, 2004

(54) Aromatic ethers and process for producing aromatic ethers

(76) Inventors: Yoshiaki Hirano, Nishinomiya-shi (JP); Hiroshi Yamamoto, Kobe-shi (JP); Takafumi Kubo, Suita-shi (JP); Hideaki Nagano, Himeji-shi (JP); Mitsuhiro Kitajima, Suita-shi (JP); Hiroko Yamaguchi, Osaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 10/626,573

(22) Filed: Jul. 25, 2003

(30) Foreign Application Priority Data
Jul. 26, 2002 (JP) 2002-217744 (PAT.
Aug. 27, 2002 (JP) 2002-247284 (PAT.
Apr. 3, 2003 (JP) 2003-100529 (PAT.
Apr. 25, 2003 (JP) 2003-122646 (PAT.
Jun. 4, 2003 (JP) 2003-159516 (PAT.

(51) Int. Cl. C07C 41/03

(52) U.S. Cl. 568/662

(57) ABSTRACT

According to a production process, aromatic ethers are producible by reacting phenols with an oxirane compound with use of an anion exchange resin as a catalyst. According to another production process, aromatic ethers having an alcoholic hydroxyl group are producible by a crystallization-purification step of using a solvent having a solubility parameter ranging from 7.5 to 12.5 for purification by crystallization. Further, according to still another production process, producible are aromatic ethers having an alcoholic hydroxyl group, wherein the content of a metal in the aromatic ethers is less than 100 ppm by mass, and the content of a halogen element in the aromatic ethers is less than 100 ppm by mass.

### BACKGROUND OF THE INVENTION

1. Field of the Invention

This invention relates to a process for producing aromatic ethers based on phenols and oxirane compound, and further pertains to a production process including a step of purifying aromatic ethers by crystallization. Furthermore, this invention relates to aromatic ethers having an alcoholic hydroxyl group.

2. Description of the Related Art

Aromatic ethers such as β-phenoxyethanols are utilized in various fields in many ways because the aromatic ethers have alcoholic hydroxyl groups in molecules thereof. The aromatic ethers is a kind of glycol ether, and is a solvent exhibiting excellent properties in working environments because of its high-boiling point. Further, the aromatic ethers are used as significant raw materials of various chemicals such as polyester materials, polyurethane materials, and (meth)acrylate materials by utilizing the action of alcoholic hydroxyl groups in molecules thereof.

In case that the aromatic ethers have phenolic hydroxyl groups in molecules thereof, such aromatic ethers are particularly widely used in the fields of cosmetic preparations, pharmaceutical preparations, and fragrant materials by utilizing bactericidal action of the phenolic hydroxyl groups. For instance, it has been found that the aromatic ethers having phenolic hydroxyl groups are useful as dermatologic preparations with safe use. Further, there is known use of the aromatic ethers as compositions of resists for integrated circuits having good image resolution, focal depth, and developability, and excellent properties in the aspect of sensitivity, resist-pattern sectional configuration, and storage stability, as well as compositions for cationic electro-deposition paint.

Generally, reaction rate of the aromatic ethers is extremely slow, and generation of byproduct is large if the aromatic ethers are synthesized in the absence of a catalyst. In view of this, the aromatic ethers are generally produced with use of a catalyst.

For instance, there is known a process for synthesizing β-phenoxyethanols contained in the aromatic ethers with use of an alkali metal salt and a quantitative amount of water (see Japanese Examined Patent Publication No. 39-30272). Use of a quantitative amount of water not only hinders efficient use of oxirane compound but also results in a large quantity of industrial waste water. Particularly, many kinds of phenols have bactericidal action, and if waste water contains unreacted phenols therein, it is difficult to carry out activated sludge process of the waste water.

For the aforementioned reasons, there is a demand for a non-waterborne process as a production process of the aromatic ethers in place of a waterborne process as disclosed in the above Japanese publication.

Examples of the non waterborne process of producing the aromatic ethers include reaction with use of a catalyst consisting of halogenated phosphonium salt (or a tertiary phosphine) and a halogenated alkyl (see Japanese Examined Patent Publication No. 50-654) and reaction in the presence of halogenated trialkylbenzyl ammoniums (see Japanese Examined Patent Publication No. 49-33183).

In the above approaches, it is a general practice to carry out excessive addition reaction of oxirane compound to phenols in order to suppress generation of unreacted phenols. Such a reaction, however, gives rise to increase of impurities in which oxirane compound is excessively added. Thereby, purity of the aromatic ethers having a desired structure may be lowered.

As a process for synthesizing the aromatic ethers having phenolic hydroxyl groups, there is known a method for adding oxirane compound to multivalent phenols in the presence of transition metal ions such as iron ions as a catalyst (see Dutch Patent No. 6600198).

A review of the present inventors reveals, however, that it is likely that quinones may be generated according to the above method because raw material multivalent phenols (catechol) are oxidized by oxygen, which exists in a slight amount in the reaction system, owing to the existence of the transition metal ions as a catalyst. The quinones are turned into so-called quinhydrones with phenols. Since quinhydrones are a factor which gives rise to colored the aromatic ethers, generation of quinones (quinhydrones) adversely affects purification step following the reaction step, which is not advantageous from the industrial viewpoint.

Generally, quinhydrone is a molecular compound consisting of hydroquinone as a multivalent phenol and p-benzoquinone as an oxidation product of hydroquinone. Quinhydrones in this specification indicate molecular compounds consisting of multivalent phenols and quinones as oxidation products thereof.

Japanese Examined Patent Publication No. 54-1291 discloses a process for adding ethyleneoxide to multivalent phenols with use of water as a solvent and with use of a halogenated quarternary ammonium compound or a halogenated quarternary phosphonium compound as a catalyst. A review of the present inventors, however, reveals that it is difficult to selectively synthesize the aromatic ethers having phenolic hydroxyl groups according to this process.

Further, there is generally used a distillation process in purifying the aromatic ethers having the aforementioned structure. Distillation of the aromatic ethers, however, requires a high degree of vacuum and a high temperature. Accordingly, in case of using multivalent phenols as a raw material, for example, it is highly likely that unreacted multivalent phenols are oxidized to quinones. Since the quinones may be turned into quinhydrones as mentioned above, colored aromatic ethers may be generated after purification. Since such quinones have sublimation property, it is difficult to remove the quinones by distillation.

Furthermore, the aromatic ethers are in a solid state at normal temperature. Accordingly, it is required to keep the sites of a distillation apparatus in contact with the aromatic ethers warm during evaporation for purification in order to keep the aromatic ethers in a liquid state, which requires enormous quantity of energy.

In view of the above, it is an object of this invention to provide a process for producing aromatic ethers having a desired structure in good selectivity while suppressing generation of byproduct, with use of phenols and oxirane compound as raw materials. It is another object of this invention to provide a process for efficiently and stably producing aromatic ethers in high purity while suppressing coloration of purified product, as well as suppressing energy consumption in a purification step. It is still another object of this invention to provide aromatic ethers having reduced content of impurities that may likely to give rise to deterioration in property as material.

### SUMMARY OF THE INVENTION

According to an aspect of this invention, the process of this invention has a feature in producing aromatic ethers by reacting phenols with oxirane compound under the presence of an anion exchange resin as a catalyst.

Use of the anion exchange resin as a catalyst makes it possible to produce aromatic ethers in high yield by raising catalytic activity. Further, according to the process of this invention, since reaction is carried out under a reaction condition substantially without water, the aromatic ethers are produced with less loss of oxirane compound and with less generation of waste water.

In case that the anion exchange resin is solid, the target compound (aromatic ethers) can be easily separated from the catalyst, and accordingly, the catalyst is repeatedly usable. Furthermore, even if the anion exchange resin is dissolved in a reaction solvent, the anion exchange resin can be taken out from the reaction system by a re-precipitation process. Thus, the catalyst is also repeatedly usable. According to the process of this invention, since the catalyst can be easily separated, a step of neutralizing the catalyst after the reaction is omitted, thereby eliminating increase of salts of unreacted phenols and salts of impurities. Furthermore, since the content of unreacted phenols is lowered, excessive use of oxirane compound is avoided, thereby leading to less generation of byproduct and securing good selectivity in generation of aromatic ethers having a desired structure.

According to another aspect of this invention, the process of this invention comprises a purification step by crystallization with use of a solvent having a solubility parameter ranging from 7.5 to 12.5. According to this process, producible are aromatic ethers having an alcoholic hydroxyl group or aromatic ethers having a phenolic hydroxyl group and an alcoholic hydroxyl group.

Use of a solvent having the solubility parameter in the above predetermined range as a crystallization solvent for purification makes it possible to suppress energy consumption in the purification step and to suppress generation of substance that induces coloring of aromatic ethers. Thereby, aromatic ethers in high purity are producible efficiently and stably.

According to still another aspect of this invention, this invention is directed to aromatic ethers having an alcoholic hydroxyl group with the content of a metal of less than 100 ppm (the unit is mass, hereinafter, the unit is the same unless otherwise specified) and the content of halogen element of less than 100 ppm. The metal and halogen may induce deterioration in property of aromatic ethers as material. The aromatic ethers of this invention are free of a problem regarding deterioration in property as material which is attributable to the existence of the metal and halogen because both the metal content and halogen content are low in the aromatic ethers of this invention.

### DETAILED DESCRIPTION OF THE PREFERRED EMBODIMENTS OF THE INVENTIOIN

The aromatic ethers of this invention are compounds producible by reacting phenols with oxirane compound, both of which will be described later, and include compounds having both an alcoholic hydroxyl group and a phenolic hydroxyl group, and compounds having an alcoholic hydroxyl group but without a phenolic hydroxyl group. The inventive aromatic ethers may be producible according to a known technique, which will be described in the section of the second production process, by reacting alkylene carbonates, halogenated alkanols or multivalent alcohols with phenols, in addition to the reaction of phenols with oxirane compound.

First, described is a process for producing aromatic ethers by reacting phenols with oxirane compound. Hereinafter, this process is called as “first production process”.

Phenols used in the first production process as a raw material may be a solid or a liquid. There is no constraint regarding the form of material (type of packing) and its purity.

The phenols used throughout the present specification are aromatic compounds in each of which at least one hydroxyl group having a property inherent to phenol (hereinafter, called as “phenolic hydroxyl group”) is contained in a molecule thereof. Aromatic compounds are compounds each having an aromatic ring. Aromatic ring includes: non-benzoic aromatic ring such as cyclopentadiene ring; benzene ring; condensed aromatic ring such as naphthalene ring, anthracene ring and pyrene ring; and heterocyclic aromatic rings in which at least one carbon atom in non-benzoic aromatic ring, aromatic ring or condensed aromatic ring is substituted by a hetero atom such as an oxygen atom, a nitrogen atom, or a sulfur atom (such as pyrrole ring, pyridine ring, thiophene ring, and furan ring).

Monovalent phenols include phenol: phenols having a hydrocarbon substituent such as o-, m-, or p-cresol, o-, m-, or p-ethylphenol, o-, m-, or p-t-butylphenol, o-, m-, or p-octylphenol, 2,3-xylenol, 2,6-xylenol, 3,4-xylenol, 3-5-xylenol, 2,4-di-t-butylphenol; phenols having a substituent group such as an aromatic substituent or an aromatic ring e.g. o-, m-, or p-phenylphenol, p-α-cumyl)phenol, and 4-phenoxyphenol; phenols having an aldehyde group such as o-, m-, or p-hydroxybenzaldehyde; phenols having a substituent group with an ether linkage such as guaiacol and guaethol; phenols having a substituent group such as a hydroxyl group with a property inherent to alcohol (hereinafter, called as “alcoholic hydroxyl group”) e.g. p-hydroxyphenethyl alcohol; phenols having a substituent group with an ester linkage such as p-hydroxy benzoic methyl, p-hydroxyphenylacetic acid methyl ester, and heptylparaben; phenols having a halogen group such as 2,4,6-trichlorophenol, and 2-amino-4-chlorophenol; phenols having a nitro group such as o- or p-nitrophenol; phenols having a substituent group with a nitrogen atom such as aminophenol, 2,4-6-tris (dimethylamino)phenol, and p-hydroxyphenylacetamide; and α-naphthol, and β-naphthol. Among these, phenol and cresol are preferred.

Multivalent phenols include: bivalent phenols including catechols (such as catechol, protocatechuic acid, chloracetylpyrocatechin, adrenalone, adrenaline, apomorphine, urushiol, tiron, phenylfluorone), resorcinols (such as resorcinol, orcinol, hexylresorcine), and hydroquinones (such as 2,3,5-trimethylhydroquinone, 2-t-butylhydroquinone, homogentistic acid ester); trivalent phenols including pyrogallols (such as pyrogallol, 2,3,4-trihydroxybenzophenone, lauryl gallate, ester gallate, and purpurogallin), phloroglucins (such as phloroglucin), and oxyhydroquinones (such as oxyhydroquinone); bisphenols such as bis(3,5-dimethyl-4-hydroxyphenyl)sulfone, bis(4-hydroxyphenyl)sulfone, 2,2′-methylenebis(4-methyl-6-t-butylphenol), 2,2′-methylenebis(4-ethyl-6-t-butylphenol), 4,4′-thiobis(3-methyl-6-t-butylphenol), 4,4′-butylidenebis(4-methyl-6-t-butylphenol), 3,9-bis(1,1-dimethyl-2(β-(3-t-butyl-4-hydroxy-5-methylphenol)propionyloxy)ethyl-2,4,8,10-tetraoxaspiro(5,5)undecane, aluminon, atranolin, erythrine, catechin, epicatechin, isocarthamin, curcumin, coclaurine, cyanidin, syringidin, stilbestrol, ester tannate, bisphenol A, bisphenol S, bisphenol Z, bisphenol fluorene, biscresol fluorene, phenol red, phloridzin, hexestrol, hematoxylin, pelargonidin, morin, and lecanoric acid; hydroxynaphthalenes such as 1,4-dihydroxynaphthalene, carbonyl-J acid, (R)-1,1′-bi-2-naphthol, (S)-1,1′-bi-2-naphthol, Eriochrome Black T, α-binaphthol, β-binaphthol, and γ-binaphthol; hydroxyanthracenes or hydroxyanthraquinones such as 1-4-dihydroxyanthraquinone, leuco-1,4-diaminoanthraquinone, leuco-1,4-dihydroxyanthraquinone, anthrahydroquinone, alizarin, Alizarine S, emodin, quinizarin, kermesic acid ester, acidic anthraquinone dye (such as Alizarine Saphirol B), purpurin, and purpuroxanthin; citrazinic acid; 1,1,3-tris(2-methyl-4-hydroxy-5-t-butylphenyl)butane; 1,3,5-trimethyl-2,4,6-tris(3,5-di-t-hydroxybenzyl)benzene; and high-molecular mltivalent phenols such as polyphenols, novolak resin, resol resin, atromentin; anthraquinone dye (vat dye purple), usnic acid, dehydrourushiol, echinochrome, orsellinic acid ester, carthamin, acidic mordant dye (such as Diamond Black F, Chrome Fast Navy Blue B; Palatin Fast Blue), dihydrophenylalanine ester, gyrophoric acid ester, delphinidin, vitamin P, fluorescein, and polyporic acid.

Among the above multivalent phenols, preferred materials for producing the aromatic ethers having a phenolic hydroxyl group include catechols, resorcinols, and hydroquinones. More preferred ones are catechol, resorcinol, and hydroquinone. Preferred materials for producing the aromatic ethers substantially free of a phenolic hydroxyl group as a residue include bisphenols. More preferred ones are bisphenol A, bisphenol S, bisphenolfluorene, and biscresolfluorene.

The oxirane compound as the other material for producing the aromatic ethers is a compound in which at least one epoxy group (tricyclic ether) is contained in a molecule thereof. The preferable oxirane compound includes: aliphatic alkylene oxides such as ethylene oxide, propylene oxide, isobutylene oxide, 1,2-butylene oxide, 2,3-butylene oxide, and pentylene oxide; aromatic alkylene oxides such as stylene oxide; and cyclohexane oxide. The oxirane compounds may be used solely or in combination of two or more thereof. Among the oxirane compounds, preferred are aliphatic alkylene oxides having 2 to 4 carbon atoms such as ethylene oxide, propylene oxide, isobutylene oxide, and 2,3-butylene oxide.

The structure of the aromatic ethers of this invention is represented by the following general formula (1).

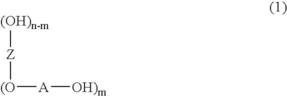

wherein Z is a hydrocarbon residue including an aromatic ring. Specifically, Z is a hydrocarbon residue including a non-benzoic aromatic ring, a benzene ring, a condensed aromatic ring, and a heterocyclic aromatic ring, which are mentioned in the description regarding phenols. OH and O-A-OH are each a group for substituting a hydrogen atom on Z, n is an integer of not smaller than 1 and not larger than the maximum number of hydrogen atoms capable of substituting on Z, and m is an integer of not smaller than 1 and not larger than n. A is represented by the general formula (2).

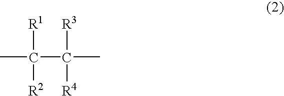

wherein R¹ through R⁴ are each independently a hydrogen atom, an alkyl group, or an aryl group. In case that R¹ through R⁴ are alkyl group and/or aryl group, R¹ through R⁴ may include various substituent groups. Alternatively, R¹ (or R²) and R³ (or R⁴) may constitute a cyclic structure (e.g. 5-membered ring or 6-membered ring).

The hydroxyl group bound to the aromatic ring in Z in the above formula (1) is “phenolic hydroxyl group” in the present specification, and the hydroxyl group bound to A is “alcoholic hydroxyl group” in the present specification.

The structure of the phenols is represented by the following general formula (3).

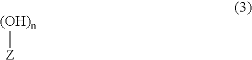

wherein Z and n are the same as in the formula (1), and OH is a group for substituting a hydrogen atom on Z.

Among the phenols, preferred are compounds in which Z is a phenyl group and n=1 (phenol), Z is a methyl phenyl group and n=1 (cresol), Z is a phenyl group and n=2 (catechol, resorcinol, and hydroquinone), and Z is a hydrocarbon residue represented by the following formulae (4) through (7) and n=2 {bisphenol A [as represented by formula (4)], bisphenol S [as represented by formula (5)], bisphenol fluorene [as represented by formula (6)], and biscresol fluorene [as represented by formula (7)]}, respectively.

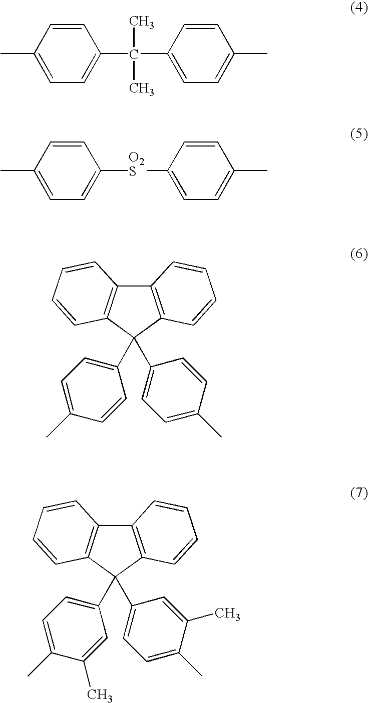

The structure of the oxirane compound is represented by the following general formula (8).

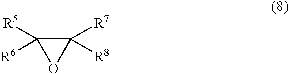

wherein R⁵ through R⁸ are each independently a hydrogen atom, an alkyl group, or an aryl group. In case that R⁵ through R⁸ are alkyl group and/or aryl group, R⁵ through R⁸ may include various substituent groups. Alternatively, R⁵ (R⁶) and R⁷ (R⁸) may constitute a cyclic structure (e.g. 5-membered ring or 6-membered ring).

Among the oxirane compounds, preferred are compounds: in which R⁵ through R⁸ are each a hydrogen atom (ethylene oxide); either one of R⁵ through R⁸ is a methyl group, and the other three are each a hydrogen atom (propylene oxide); R⁵ and R⁶ are each a methyl group, and R⁷ and R⁸ are each a hydrogen atom (isobutylene oxide); R⁵ and R⁷ are each a methyl group, and R⁶ and R⁸ are each a hydrogen atom (2,3-butylene oxide), respectively.

The catalyst used in the first production process is an anion exchange resin. Use of anion exchange resin as a catalyst to synthesize aromatic ethers based on multivalent phenols and oxirane compound is effective in producing aromatic ethers having a desired structure as a target compound while suppressing generation of byproduct. In other words, aromatic ethers having a desired structure can be produced with improved selectivity. Hereinafter, a property capable of selectively producing aromatic ethers having a desired structure is sometimes referred to as “selectivity (or reaction selectivity)”.

Anion exchange resin is a polymer compound having anion exchangeability. The anion exchange resin may be the one dissoluble in a reaction solvent (to be described later), or may be the one which does not dissolve in a solvent and remains as a solid. Specifically, the anion exchange resin can take various forms during the above reaction such as a slurry form with a reaction solution, or a solid form in a reaction solution, as well as a uniformly dissolved state in a reaction solution. In case that the anion exchange resin exists as a solid, the anion exchange resin can take various forms such as granules, particles, powders, or a state that the resin is supported on a base.

In view of handling that the anion exchange resin is taken out of the reaction system for reuse after the reaction, preferably, the anion exchange resin exists as a solid without being dissolved in a reaction solvent. However, the anion exchange resin dissoluble in a reaction solvent may be reusable by recovering the resin by a known re-precipitation process.

The anion exchange resin has a main chain moiety and an anion exchange group as essential components. Preferably, the anion exchange resin has a cross-linking site. Examples of the anion exchange group are the ones having respective structures of tertiary amines, quarternary ammonium salts, tertiary phosphines, or quarternary phosphonium salts. Among these, preferred are the ones having structures of quarternary ammonium salts and quarternary phosphonium salts. Further preferably, the anion exchange group may have a structure excellent in heat resistance. Specifically, it is preferable that the anion exchange group may have the following first or second structure.

The first structure is such that the anion exchange group has a cyclic structure. Preferred cyclic structures are 5-membered rings and 6-membered rings. A more preferred cyclic structure is a 5-membered ring. Particularly, in case that the anion exchange group has a structure of quaternary ammonium salt, it is preferable that the anion exchange group has a piperidine skeleton or a pyridine skeleton. Among the anion exchange resins having a structure of cyclic quaternary ammonium salt, recommendable are the ones that can be synthesized based on diallyldimethyl ammonium chloride in the aspect of feasibility in formation of the resin.

The second structure is such that the anion exchange group has a structure bound to a main chain moiety by way of an alkylene chain having 4 or more carbon atoms.

The structure of a quaternary ammonium salt or a quaternary phosphonium salt in the anion exchange group has a feature that an anion makes a pair with a cationic hetero atom. Species of anion in an initial stage of the anion exchange resin in this invention are not specifically limited. Examples of anions used in this invention are: hydroxide ion; halide ions such as fluorine, chlorine, bromine, and iodine; anions of organic acid (such as carboxylic acid anions and phenoxy anions): and anions of inorganic acid. Examples of anions of inorganic acid include sulfuric acid ion, sulfurous acid ion, hydrogen sulfite ion, phosphorous acid ion, boric acid ion, cyanide ion, carbonic acid ion, thiocyanic acid ion, nitric acid ion, phosphoric acid ion, hydrogen phosphate ion, and metalate ion (such as molybdic acid ion, tungstic acid ion, tungstophosphoric acid ion, metavanadic acid ion, pyrovanadic acid ion, hydrogen pyrovanadic acid ion, niobic acid ion, and tantalic acid ion). Among these, preferred are anions of various organic acids, hydroxide ion and halide ions.

Both of low-molecular anion exchange resins and high-molecular anion exchange resins are usable as the anion exchange resin. In case of using a low-molecular anion exchange resin, the low-molecular anion exchange resin has preferably a molecular weight of 500 or more, and more preferably 1,000 or more in light of removability of the catalyst for recovery and reusability after reaction of phenols with oxirane compound is completed.

Specific examples of the anion exchange resin include the ones in which an anion exchange group has the second structure such as “DIAION® TSA1200” manufactured by Mitsubishi Chemical Corporation.

Although the heat resistance is little lowered compared with the aforementioned product, usable are the following commercially available anion exchange resins such as: “DIAION® PA300 series (e.g. PA306)”, “DIAION® PA400 series (e.g. PA406)”, “DIAION® HPA25, 75“(all of which are manufactured by Mitsubishi Chemical Corporation”; “DOWEX®” (SBR, SBR-P-C, SAR, MSA-1, MSA-2, 22, MARATHON® A, MARATHON® ALB, MARATHON® A2, MONOSPHERE® 550A) (all of which are manufactured by Dow Chemical Company); “Duolite®” (A113, A113LF, A113 MB, A109D, A116, A116LF, A161TRSO4, A162LF, A368S, A378D, A375LF, A561, A568K, A7), and “AMBERLITE®” (IRA-400T, IRA-410) (all of which are manufactured by Rohm and Haas Company).

Phenols and oxirane compound may be reacted with each other with or without use of a solvent. In case of using a solvent, a solvent such as water, an organic solvent, and a mixture of water and an organic solvent may be used. Since it is difficult to carry out activated sludge process for waste water containing phenols, it is preferable to use a solvent other than water.

Reaction of phenols with oxirane compound can be advantageously carried out with use of the anion exchange resin as a catalyst substantially in the absence of water. Accordingly, water is not required in the reaction. The phrase “substantially in the absence of water” means the content of water relative to the total content of raw materials (phenols and oxirane compound) is less than 1% by mass, and more preferably, less than 1,000 ppm by mass.

Specific examples of solvents usable in the first production process include: alcohols having 1 to 6 carbon atoms such as methanol, ethanol, n-propanol, 2-propanol, n-butanol, hexanol, and cyclohexanol; glycol ethers having 3 to 6 carbon atoms such as ethylene glycol monomethyl ether, ethylene glycol monoethyl ether, and ethylene glycol monobutyl ether; ethers having 2 to 6 carbon atoms such as tetrahydrofurane and dioxane. Also, usable are aliphatic hydrocarbons such as pentane, hexane, and heptane; aromatic hydrocarbons such as benzene, toluene, and xylene; alicyclic hydrocarbons such as cyclohexane; ketones such as acetone, methylethyl ketone, methylisobutyl ketone, methyl isopropyl ketone, and cyclohexanone; esters such as ethyl acetate and butyl acetate; halogenated hydrocarbons such as dichloromethane, 1-2-dichloroethane; glycols such as ethylene glycol; pyridine; acetonitrile; dimethyl sulfoxide; dimethyl formamide; and ethylene carbonate.

The solvent used in the first production process may preferably have a solubility parameter ranging from 7.0 to 20.0. Using the solvent having a solubility parameter in the predetermined range in combination with the catalyst for reaction of multivalent phenols with oxirane compound is advantageous in carrying out the reaction while leaving a part of the phenolic hydroxyl group in the multivalent phenols unreacted depending on the selected reaction condition, thereby remarkably enhancing selectivity in generation of desired aromatic ethers having a phenolic hydroxyl group. Although a reason for such enhanced reaction selectivity has not been elucidated, it is conceived that as far as the solubility parameter of the solvent lies within the above range, the solubility parameter of the solvent is approximate to the solubility parameter of the main chain moiety of the anion exchange resin as a catalyst. It is conceived that the approximation between the solubility parameters contributes to improvement in reaction selectivity of synthesizing aromatic ethers having a desired structure.

“Solubility parameter” (δ) in this invention is an index on the scale of blendability between or among solutions, which is expressed in terms of a square root of a cohesive energy density according to a theory regarding regular solution and is represented by the formula (9).

δ=(ΔE^{v}/V)^{1/2} (9)

wherein V denotes molar volume of the solvent (unit: cm³/mol), and ΔE^{v} denotes heat of evaporation (unit: cal/mol) of the solvent at 25° C.

Among the solvents usable in the first production process, some of the examples of the solvents having a solubility parameter in the aforementioned range are pentane (δ=7.0), hexane (δ=7.3), heptane (δ=7.4), cyclohexane (δ=8.2), methyl isobutyl ketone (δ=8.4), butyl acetate (δ=8.5), o-xylene (δ=8.8), p-xylene (δ=8.8), toluene (δ=8.9), tetrahydrofuran (δ=9.1), ethyl acetate (δ=9.1), benzene (δ=9.2), methylethylketone (δ=9.3), dichloromethane (δ=9.7), 1,2-dichloroethane (δ=9.8), cyclohexanone (δ=9.9), acetone (δ=9.9), 1,4-dioxane (δ=10.0), pyridine (δ=10.7), ethyleneglycol monomethyl ether (δ=11.4), 1-butanol (δ=11.4), 2-propanol (δ=11.5), acetonitrile (δ=11.9), dimethyl sulfoxide (δ=12.0), dimethylformamide (δ=12.1), ethanol (δ=12.7), methanol (δ=14.5), ethylene glycol (δ=14.6), and ethylene carbonate (δ=14.7). The solubility parameters of the respective solvents are disclosed, for instance, in “Chemical Reviews” (A. F. M. Burton, 1975, Vol. 75, No. 6, pp.731-753).

In the first production process, the aforementioned solvents may be used solely or in combination of two or more thereof. In case of admixing two or more of the solvents, the solubility parameters of the respective solvents are not specifically limited, as far as the solubility parameter of the mixed solvent lies within the aforementioned range. The solubility parameter δₘᵢₓ of the mixed solvent is calculated according to the formula (10).

δₘᵢₓ(x₁V₁δ₁+x₂V₂δ₂+. . . xₙVₙδₙ)/(x₁V₁+x₂V₂+. . . xₙVₙ) (10)

wherein n denotes the kind of the solvent to be mixed, and x, V, δ denote the molar fraction, molar volume, and solubility parameter of each solvent, respectively.

The productivity of the aromatic ethers is likely to be deteriorated if the solubility parameter of the solvent falls below the lower limit of the aforementioned range because as the solubility parameter of the solvent is lowered, the solubility of the multivalent phenols is lowered and the concentration thereof is lowered. A preferred lower limit of the solubility parameter range of the solvent is 8.0, and a more preferred lower limit thereof is 8.5. On the other hand, the selectivity of the resulting aromatic ethers (yield of aromatic ethers having a phenolic hydroxyl group) is likely to be deteriorated if the solubility parameter of the solvent exceeds the upper limit of the aforementioned range because as the solubility parameter of the solvent is raised, increased is a difference in solubility parameter between the hydrocarbon residue in the catalyst and the solvent. A preferred upper limit of the solubility parameter range of the solvent is 11.5, and a more preferred upper limit thereof is 10.5. Among the above solvents, particularly preferred solvents are, for instance, butyl acetate (δ=8.5), toluene (δ=8.9), methylethylketone (δ=9.3), and 1,4-dioxane (δ=10.0).

In case of using the solvent having a solubility parameter in the above range as a reaction solvent in producing aromatic ethers having a phenolic hydroxyl group by reacting multivalent phenols as the phenols with the oxirane compound, use of quarternary salts such as quarternary ammonium salt and/or quarternary phosphonium salt as a catalyst, as well as the anion exchange resin also makes it possible to yield the aromatic ethers in good reaction selectivity. In other words, this process is directed to a process for producing aromatic ethers by reacting phenols with oxirane compound with use of a quaternary salt as a catalyst in the presence of a solvent having a solubility parameter in the range from 7.0 to 20.0.

The quarternary salts as a catalyst used in this invention are not specifically limited, as far as they are quarternary ammonium salts and/or quarternary phosphonium salts. More specifically, preferred catalysts are: quarternary ammonium salts such as tetramethyl ammonium chloride, tetramethyl ammonium bromide, tetrabutyl ammonium chloride, tetrabutyl ammonium bromide, tetraoctyl ammonium chloride, tetraoctyl ammonium bromide, tetraoctyl ammonium iodide, tributylmethyl ammonium chloride, trioctylmethyl ammonium chloride, trilaurylmethyl ammonium chloride, benzyltributyl ammonium chloride, phenyltrimethyl ammonium chloride; and quarternary phosphonium salts such as tetramethyl phosphonium chloride, tetramethyl phosphonium bromide, tetrabutyl phosphonium chloride, tetrabutyl phosphonium bromide, tetraoctyl phosphonium chloride, tetraoctyl phosphonium bromide, tetramethyl phosphonium iodide, tributylmethylmethyl phosphonium chloride, trioctylmethyl phosphonium chloride, trilaurylmethyl phosphonium chloride, and benzyltributyl phosphonium chloride.

Among the above quarternary ammonium salts and/or quarternary phosphonium salts, preferred are the ones having a hydrocarbon residue with 4 or more carbon atoms, and more preferred are quarternary ammonium salts and/or quarternary phosphonium salts represented by the following formula (11) in order to obtain improved reaction selectivity.

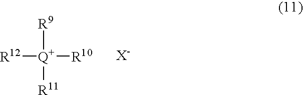

wherein Q is a nitrogen atom or a phosphorous atom, R⁹, R¹⁰, R¹¹, R¹² are each a hydrocarbon residue, at least one of R⁹, R¹⁰, R¹¹, R¹² is a hydrocarbon residue having 4 or more carbon atoms, and X is a counter ion.

Examples of a hydrocarbon residue having 4 or more carbon atoms are aliphatic alkyl group having 4 or more carbon atoms, alicyclic alkyl group, or aryl group. Aliphatic alkyl group may include a branched chain. Alicyclic alkyl group or aryl group may be the one in which at least one hydrogen on the ring is substituted by a hydrocarbon residue. Further, a hydrocarbon residue having 4 or more carbon atoms may be a polymer chain. Preferably, at least two of R⁹ through R¹² may be a hydrocarbon residue having 4 or more carbon atoms, more preferably, at least three of R⁹ through R¹² may be a hydrocarbon residue having 4 or more carbon atoms, and particularly preferably, all of R⁹ through R¹² may be a hydrocarbon residue having 4 or more carbon atoms. In case that at least one of R⁹ through R¹² is a hydrocarbon residue having 4 or more carbon atoms, other hydrocarbon residue(s) (hydrocarbon residue(s) having 3 or less carbon atoms) may be, for example, aliphatic or alicyclic alkyl group having 3 or less carbon atoms. As an altered form, two of R⁹ through R¹² may form a ring structure (e.g. 5-membered ring or 6-membered ring).

Examples of anions corresponding to X⁻ include various anion species illustrated as anions in the anion exchange resin. Among the anion species, anions of various organic acids, hydroxide ion, and halide ions are preferred.

In case of using the quarternary salt as a catalyst, it is preferable to react multivalent phenols and oxirane compound as starting materials with each other by way of a quarternary salt in a state that these ingredients are dissolved or dispersed in a solvent (in liquefied state) to synthesize the aromatic ethers of this invention. In view of this, recommendable catalysts are the ones dissoluble in the avobe-mentioned solvent, namely, homogeneous catalysts. It is desirable to use the catalyst in which the hydrocarbon residue having 4 or more carbon atoms in the compounds R⁹ through R¹² in the above formula (11) has carbon atoms of 22 or less (preferably 12 or less) in light of solubility in a solvent.

Particularly preferred examples of quarternary ammonium salts and/or quarternary phosphonium salts which satisfy the above requirements are tetrabutyl ammonium bromide, tributylmethyl ammonium chloride, trioctylmethyl ammonium chloride, trilaurylmethyl ammonium chloride, benzyltributyl ammonium chloride, tetraoctyl ammonium chloride, tetrabutyl phosphonium bromide, tributylmethyl phosphonium chloride, trioctylmethyl phosphonium chloride, trilaurylmethyl phosphonium chloride, benzyltributyl phosphonium chloride, and tetraoctyl phosphonium chloride.

As far as the solvent has a solubility parameter in the above range, the solubility parameter of the solvent is approximate to the solubility parameter of the hydrocarbon residue of the quarternary salt [represented by R⁹ to R¹² in the formula (11)]. Accordingly, it is conceived that the approximation between the solubility parameters contributes to improvement in reaction selectivity of synthesizing aromatic ethers having a desired structure while leaving at least one phenolic hydroxyl group unreacted. Particularly, in case that at least one of the hydrocarbon residues represented by R⁹ to R¹² in the formula (11) is a hydrocarbon residue having 4 or more carbon atoms, reaction selectivity is further improved because the solubility parameter of the hydrocarbon residue and the solubility parameter of the solvent is further approximated to each other.

As mentioned above, in case of using a solvent having a specific solubility parameter as a reaction solvent in producing the aromatic ethers by reacting multivalent phenols with oxirane compound while leaving at least one phenolic hydroxyl group unreacted, use of the quarternary salt, as well as the anion exchange resin makes it possible to yield the aromatic ethers in good reaction selectivity. It is, however, recommended to use the anion exchange resin as a catalyst considering that the catalyst is removed for reuse from the reaction system after the reaction.

The reaction manner in the first production process is not specifically limited. The reaction may be implemented in a batch manner or continuously. In case of continuous reaction, the catalyst can be brought to a suspension state in a reaction vessel. Also, the reaction can be carried out by allowing the raw materials to pass while using the catalyst as a fixed bed. It is preferable that the raw materials are in a uniformly blended state during the reaction. Alternatively, the materials may be divided into two layers as far as the reaction is executable.

The amount of the raw material for feeding, the amount of the catalyst, the amount of the solvent, the reaction time, and the reaction temperature are not specifically limited as far as the reaction is executable. These conditions can be optionally selected depending on the structure of aromatic ethers to be synthesized.

In case of producing aromatic ethers having a phenolic hydroxyl group using multivalent phenols as phenols, it is desirable to adopt the following conditions. Specifically, the feeding amount of the raw material is preferably such that the amount of oxirane compound ranges from 0.1 to 2.0 in molar ratio to the amount of phenolic hydroxyl group to which the oxirane compound is to be added in the multivalent phenols, more preferably from. 0.5 to 1.5, and furthermore preferably from 0.9 to 1.2.

On the other hand, it is recommended to adopt the following conditions in case of producing aromatic ethers by reacting phenols with oxirane compound with least residue of phenolic hydroxyl group with use of mono- or multi-valent phenols as phenols. Specifically, the feeding amount of the raw material is preferably such that the amount of oxirane compound is at least 0.9 in molar ratio to the amount of phenolic hydroxyl group in the multivalent phenols, and more preferably 0.95 or more, and furthermore preferably 1.0 or more. A preferred upper limit of the amount of oxirane compound to the amount of phenolic hydroxyl group in the multivalent phenols is 5 in molar ratio, more preferably 2, furthermore preferably 1.5, particularly preferably 1.2, and most preferably 1.05.

It is recommended that the amount of the catalyst, the amount of the solvent, the reaction time, and the reaction temperature fall within the below-mentioned ranges, respectively, regardless of the structure of aromatic ethers to be produced.

The amount of the catalyst preferably ranges from 1 to 70 vol. % relative to the total volume of the reaction solution including the catalyst, and more preferably from 5 to 30 vol. %.

The amount of the solvent preferably ranges from 0 to 5 in mass ratio relative to the amount of multivalent phenols as a raw material, and more preferably from 0 to 2.5.

The reaction temperature preferably ranges from 50 to 150° C., and more preferably from 80 to 120° C. The reaction time preferably ranges from 1 to 24 hours considering the productivity of aromatic ethers, and more preferably ranges from 1 to 12 hours.

The aromatic ethers thus produced by the first production process have a high purity. Accordingly, the aromatic ethers can be supplied as a product without performing a post-process after the reaction is completed. Alternatively, it is preferable to supply the aromatic ethers as a product after removing impurities by a generally known process such as distillation, extraction, and crystallization after the reaction is completed to obtain purified aromatic ethers according to needs. Further alternatively, raw material phenols may be removed and separated by a known process such as distillation, extraction, and crystallization. As a further altered form, it is possible to use a catalyst so as convert the raw material phenols into such a state that the reaction system is substantially free of phenols and to separate a resulting product in which the phenols have been converted according to the aforementioned generally known process.

Examples of the distillation process include distillation under reduced pressure, steam distillation, molecular distillation, and extractive distillation. The distillation process is not limited thereto. Examples of the crystallization process include: (1) cooling the reaction solution after the reaction is completed; (2) adding a poor solvent relative to a target compound to the reaction solution; (3) distilling off the solvent in the reaction solution; and (4) gradually pressurizing the reaction solution. These methods may be used solely or in combination thereof.

Among the above methods, it is preferable to use a solvent in a reaction step of phenols and oxirane compound and to use the solvent in a crystallization step which follows the reaction step (common use of the solvent in the reaction step and the crystallization step). This approach is advantageous in obtaining a target compound economically because suppressed are loss of the solvent and the cost required for recovering the solvent. “Common use of the solvent” in this context means that, there is not added a solvent other than the solvent used in the reaction step, to the reaction solution obtained in the reaction step. Namely, the following approaches are embraced in “common use of the solven”: feeding the reaction solution obtained in the reaction step to the crystallization step substantially without any process intervening between the reaction step and the crystallization step; and adding the same kind of solvent as the solvent used in the reaction step to the reaction solution and feeding the mixture to the crystallization step. In any case, it is preferable that the entirety or a part of the solvent used in the reaction step may be contained in a crystallization solution used in the crystallization step.

The crystallization step may constitute one or more crystallization stages. It is preferable to carry out the crystallization step in a sealed state filled with inert gas, in place of air-released state. If the crystallization step is carried out in an air-released state, it is highly likely that oxygen may be absorbed in a mother liquor for crystallization, thereby deteriorating hue of a resultant product and making it difficult to yield high-quality product. The mother liquor after separation of precipitants is fed back to the reaction step or to the crystallization step for reuse in view of efficient use of raw material phenols, according to needs. Thereby, at least part of the crystallized mother liquor can be reused as a raw material mother liquor. According to a general practice, a plurality of crystallization stages and a solid-liquid separation step of separating precipitants from the liquid are employed, and a plurality of kinds of mother liquors (solutions containing phenols) are obtained accordingly. According to the first production process of this invention, mother liquors obtained in these optional stages can be used as raw material mother liquors in the reaction step or in the crystallization step. As mentioned above, common use of a solvent in the reaction step and the crystallization step is furthermore efficient.

As mentioned above, the aromatic ethers produced by the first production process are usable as a product after the reaction is completed even in a state that the aromatic ethers contain unreacted raw material(s) and other impurities, as far as the resultant aromatic ethers are usable depending on a purpose of use and are free from a disadvantage in use. As an altered form, it is preferable to take out the aromatic ethers from the reaction solution, purify the same according to a known purification process, and to supply the purified aromatic ethers as a product. In the latter case, it is recommended to adopt a purification step by crystallization (hereinafter, called as “crystallization-purification step”) used in the second production process, which will be described later.

As mentioned above, the catalyst such as alkali metal salt and metal hydroxide is used in producing the aromatic ethers. If the conventional catalyst is used in producing the aromatic ethers of this invention, it is likely that a small amount of metal (e.g. alkali metal in case that the catalyst is an alkali metal salt, and a metal in a metal hydroxide in case that the catalyst is the metal hydroxide) may be intruded in the aromatic ethers as resultant products even if a catalyst removal step is carried out. Further, if a quarternary salt such as a halogenated ammonium salt and a halogenated phosphonium salt is used, it is likely that halogen (e.g. fluorine, chlorine, bromine, and iodine) may be intruded in the resultant aromatic ethers.

There is a demand for suppressing the intrusion rate of metal and halogen considering likelihood that these elements may be a cause of deteriorating material property and environmental adverse effect in the field of application of aromatic ethers. In view of this, the upper limit of the allowable intrusion rate of metal is 100 ppm, preferably 50 ppm, and more preferably 30 ppm, furthermore preferably 10 ppm, and particularly preferably 1 ppm. The upper limit of the allowable intrusion rate of halogen is 100 ppm, preferably 50 ppm, more preferably 30 ppm, furthermore preferably 10 ppm, and particularly preferably 1 ppm. Excessive intrusion of metal or halogen above these upper limits may deteriorate material properties of aromatic ethers in use.

Use of the anion exchange resin as a catalyst in the first production process is advantageous in that the content of metal and halogen (impurities) in the resultant crude product and purified product after the reaction is significantly small, and that product substantially free of metal and halogen is producible. Specifically, according to this method, producible are products with the content of metal and/or halogen of less than 100 ppm (the unit is mass, hereinafter, the unit is the same unless otherwise specified), preferably less than 50 ppm, more preferably less than 30 ppm, furthermore preferably less than 10 ppm, and particularly preferably less than 1 ppm.

The content of metal in the present specification is a value measured by an inductively coupled plasma spectrometry, and the content of halogen is a value measured by an X-ray fluorescence spectrometry.

Specifically, an inductively coupled plasma spectrometer “SPS4000” (manufactured by Seiko Instruments Inc.) is used as a spectrometer in measuring the content of metal.

An X-ray fluorescence spectrometer “PW2404′ (manufactured by Philips Japan, Ltd.) is used as a spectrometer in measuring the content of halogen. In the analysis, the qualitative analysis program installed in the spectrometer is used, and quantitative determination is carried out by comparing with standard specimens of halogen elements (fluorine, chlorine, bromine, and iodine).

If the content of halogen is below the detectable limit by the X-ray fluorescence spectrometer, an ion chromatography spectrometer “DX-500” (manufactured by Dionex Corporation) may be used. Since halogen is detectable in an ionized state according to ion chromatography, measurement is carried out after ionizing halogen according to needs. The measurement conditions are as follows.

| | |
|---|---|
| Detector: | CD-20 (electric conductivity detector) |
| Column: | AS4A-SC |
| Guard column: | AG4A-SC |
| Eluent: | 1.8 mmol/L Na₂CO₃, |
| | 1.7 mmol/L NaHCO₃ |
| Recovery solution: | 25 mmol/L H₂SO₄ |

Since the anion exchange resin is used in the first production process, the target compound can be produced efficiently with less content of impurities such as byproduct and unreacted material. Further, combined use of a solvent having a specific solubility parameter and a quarternary salt as a catalyst also makes it possible to produce the target compound efficiently while suppressing the content of impurities such as byproduct and unreacted material, as is the case of using the anion exchange resin.

Furthermore, in case of using the anion exchange resin as a catalyst, it is possible to efficiently carry out purification of reaction products since it is easy to separate the catalyst from the reaction solution. Thereby, the content of unreacted phenols is suppressed as low as less than 500 ppm relative to the aromatic ethers (the unit is mass, hereinafter, the unit is the same unless otherwise specified), preferably less than 100 ppm, and more preferably less than 30 ppm. Also, the excessive addition of oxirane compound as impurities in the resultant aromatic ethers can be suppressed as low as less than 10% by mass, preferably less than 5% by mass, and more preferably less than 2% by mass relative to the content of the aromatic ethers.

Further, in case of producing aromatic ethers having a phenolic hydroxyl group as a target compound according to the first production process, the target compound can be produced in high selectivity. For instance, in case of reacting catechols (bivalent phenols) with ethylene oxide (oxirane compound), it is possible to yield 2-(2-hydroxyphenoxy)ethanol in which one phenolic hydroxyl group is left unreacted in high selectivity. The compound may preferably have a phenolic hydroxyl group with the content of not smaller than 6 mmol/g. The content of phenolic hydroxyl group left unreacted in the compound differs depending on the kind of raw material phenols and the target aromatic ethers.

Next, described is a production process including a crystallization-purification step with use of a specific solvent (hereinafter, called as “second production process”).

Aromatic ethers producible by the second production process are the ones in which an alkylene chain having an alcoholic hydroxyl group is bound to an aromatic ring by way of an ether linkage (hereinafter, such a compound is referred to as “alcoholic-hydroxyl-group-containing aromatic ethers”), or the ones in which an alkylene chain having an alcoholic hydroxyl group is bound to an aromatic ring having a phenolic hydroxyl group by way of an ether linkage (namely, aromatic ethers having a phenolic hydroxyl group and an alcoholic hydroxyl group, which is hereinafter referred to as “phenolic-hydroxyl-group-containing aromatic ethers”).

The second production process is a process for producing aromatic ethers having a high purity by dissolving raw material aromatic ethers in a solvent having a specific solubility parameter and by performing purification by crystallization. In this section, raw material aromatic ethers are those containing unreacted raw materials used for synthesizing the aforementioned alcoholic-hydroxyl-group-containing aromatic ethers or the aforementioned phenolic-hydroxyl-group-containing aromatic ethers, and impurities such as byproduct generated during the synthesis, as well as the alcoholic-hydroxyl-group-containing aromatic ethers or the phenolic-hydroxyl-group-containing aromatic ethers.

The process for producing raw material aromatic ethers is not specifically limited. A suitable synthesizing process is optionally selected from among the following production processes such as: the first production process for reacting phenols with oxirane compound under the above predetermined reaction conditions; a process for reacting dihydroxy benzene with ethylene carbonate in the presence of decarboxylase such as alkali metal carbonate, alkali metal hydroxide, or alkali earth metal hydroxide (see Japanese Unexamined Patent Publication No. 2-96545, for example); a process for reacting multivalent phenols such as catechol with alcohol such as ethylene glycol in a gaseous phase with use of an orthophosphate of a trivalent rare earth metal as a catalyst (see Japanese Unexamined Patent Publication No. 6-228038); a process for reacting bivalent phenols such as catechol, resorcin, and hydroquinone with alkylene oxide such as ethylene oxide (oxirane compound) under an alkaline atmosphere (see Japanese Examined Patent Publication No. 51-4977); a process for reacting catechol with ethylene oxide with use of iron, iron chloride, or iron sulfate as a catalyst (see Dutch Patent No. 6600198); a process for adding ethylene chlorohydrin dropwise to monosodium salt of resorcin under ethanol in a reflux condition (see “Journal of the American Chemical Society”, U.S.A., 1932, Vol. 54, pp. 1195-1196); and a process for reacting resorcin or monoalkylate of resorcin with ethylene chlorohydrin in the presence of sodium hydroxide or potassium hydroxide (see U.S. Pat. No. 205,115 depending on the kind of the starting material to be used.

In the second production process, used is a crystallization solvent having a solubility parameter ranging from 7.5 to 12.5. Using the solvent having a solubility parameter in the above range facilitates preparation of a solution in which crystals of aromatic ethers are desirably precipitated by, for example, slightly changing the state of the crystallization solution.

In the case that the solubility parameter of the crystallization solvent falls below the lower limit of the solubility parameter range, it is practically difficult or impossible to precipitate crystals since the solubility of aromatic ethers in the crystallization solvent is exceedingly small. In view of this, a preferred lower limit of the solubility parameter range of the crystallization solvent is 8.0, and more preferably 8.5. Contrary to the above, if the solubility parameter of the crystallization solvent exceeds the upper limit of the solubility parameter range thereof, it is necessary to increase the degree of change of state of the crystallization solution required for crystal precipitation because the solubility of aromatic ethers in the crystallization solvent is exceedingly large, which thereby may lead to lowering of the crystallization efficiency. In view of this, a preferred upper limit of the solubility parameter range of the crystallization solvent is 11.0, more preferably 10.0, and furthermore preferably 9.5.

The solubility parameter of the crystallization solvent used in the second production process is identical to that of the reaction solvent used in the first production process. The crystallization solvent is not specifically limited as far as the solubility parameter thereof lies with in the aforementioned range. Examples of the solubility parameter of the crystallization solvent are cyclohexane (β=8.2), butyl acetate (β=8.5), o-xylene (δ=8.8), p-xylene (δ=8.8), toluene (δ=8.9), tetrahydrofuran (δ=9.1), ethyl acetate (δ=9.1), benzene (δ=9.2), methylethylketone (δ=9.3), dichloromethane (δ=9.7), 1,2-dichloroethane (δ=9.8), cyclohexanone (δ=9.9), acetone (δ=9.9), 1,4-dioxane (δ=10.0), pyridine (δ=10.7), ethyleneglycol monomethyl ether (δ=11.4), 1-butanol (δ=11.4), 2-propanol (δ=11.5), acetonitrile (δ=11.9), dimethyl sulfoxide (δ=12.0), and dimethylformamide (δ=12.1). Among these solvents, particularly preferred are butyl acetate, xylenes (o-xylene, m-xylene, p-xylene, and mixed-xylene of two or more thereof), toluene, ethyl acetate, and methylethylketone.

In the second production process, the above solvents may be used solely or in combination of two or more thereof. When a mixed solvent composed of two or more of the solvents is used, it is recommended that the mixed solvent contains the solvent having a solubility parameter ranging from 7.5 to 12.5 in the content of 20% or more by mass, more preferably 40% or more by mass, and furthermore preferably 60% or more by mass to the mixed solvent, depending on the solubility parameters of the respective solvents composing the mixed solvent. In this case, it is preferable to regulate the solubility parameter δₘᵢₓ of the mixed solvent represented by the formula (10) in the range from 7.5 to 12.5 (preferably, 8.0 or more, more preferably 8.5 or more, and preferably 11.0 or less, more preferably 10.0 or less, and furthermore preferably 9.5 or less).

Preferably, the content of aromatic ethers as a target compound in raw material aromatic ethers to be fed to the crystallization-purification step ranges from 40 to 98% by mass. If the content of aromatic ethers as a target compound falls below the lower limit, it is likely that yield of aromatic ethers as a target compound after the purification may be lowered. In view of this, a preferred lower limit of the content of aromatic ethers as a target compound is 50% by mass, and a more preferred lower limit thereof is 60% by mass. On the other hand, if the content of aromatic ethers as a target compound exceeds the upper limit, it is likely that purification effect of aromatic ethers as a target compound may be lowered. In view of this, a preferred upper limit of the content of aromatic ethers as a target compound is 95% by mass, and a more preferred upper limit thereof is 90% by mass. Further, it is possible to perform other purification technique prior to the crystallization-purification step so as to keep the content of aromatic ethers as a target compound in the raw material aromatic ethers in the above range. Examples of such other purification technique include various known methods such as concentration, distillation, and washing.

Furthermore, it is desirable to remove the catalyst used in the reaction step from the raw material aromatic ethers prior to the crystallization-purification step. This is because there is likelihood that the catalyst may be contained in the target compound after the crystallization-purification step depending on the kind of the catalyst. A suitable catalyst removal method may be optionally selected from among various known methods such as adsorption, filtration, concentration, distillation, and washing depending on the kind of the used catalyst. The residual amount of the catalyst in the raw material aromatic ethers is preferably 1% or less by mass, more preferably 0.5% or less by mass, and furthermore preferably 0.1% or less by mass.

In the following, a desirable crystallization condition is described, taking examples of mono(hydroxyethyl) ethers of dihydroxybenzene among the aforementioned aromatic ethers. In case of crystallizing other aromatic ethers, crystallization conditions may be altered individually depending on the properties of the respective aromatic ethers. For instance, in determining the concentration of the crystallization solution and the crystallization temperature, a solubility curve (a curve representing a relation between temperature and solubility of aromatic ethers) is obtained in association with the aromatic ethers and the solvent to be used. Thus, the concentration of the crystallization solution and the crystallization temperature can be easily determined based on the solubility curve.

Mono(hydroxyethyl) ethers of dihydroxybenzene is a substance in which one phenolic hydroxyl group in dihydroxybenzenes is converted to a hydroxyethoxy group. Dihydroxybenzenes include catechol, resorcin, hydroquinone, and substituents in which at least one hydrogen atom on respective benzene rings of these compounds is substituted by a hydrocarbon residue (such as an alkyl group), a halogen atom, or its equivalent.

It is recommended to attain the concentration of the crystallization solution such that the content of mono(hydroxyethyl) ethers of dihydroxybenzene as a raw material is 0.1% or more by mass, preferably 1% or more by mass, and furthermore preferably 5% or more by mass to the total amount of the solution. If the concentration of the crystallization solution falls below the lower limit, productivity [yield of purified mono(hydroxyethyl) ethers of dihydroxybenzene] may be lowered, which necessitates recovery of a quantitative amount of the solvent. This may lead production cost rise and is not desirable economically.

On the other hand, the upper limit of the concentration of the crystallization solution is desirably 40% by mass, preferably 30% by mass, and more preferably 20% by mass. If the concentration of the crystallization solution exceeds the upper limit, it is difficult to carry out solid-liquid stirring in precipitation of crystals, which may hinder industrial exploitation. Further, as will be described later, although it is preferable to carry out crystallization while stirring the crystallization solution, the following likelihood should be considered. Namely, if the concentration of mono(hydroxyethyl) ethers of dihydroxybenzene exceeds the aforementioned upper limit, it is difficult to carry out solid-liquid stirring after precipitation of crystals of mono(hydroxyethyl) ethers of dihydroxybenzene, which may lead to failure in obtaining desirable slurry, insufficient purification, or difficulty in taking out crystals.

In case that there exist insoluble matters during heating of the crystallization solution after preparing the crystallization solution, it is preferable to carry out a separation step prior to the crystallization step to remove the insoluble matters. Various known separation methods are feasible, e.g., filtration such as filtration under reduced pressure, and pressure filtration, as well as centrifugal separation. The filter fabric and the filter used in filtration are not specifically limited as far as these filter members have a filtration capability of removing insoluble matters.

A desirable temperature for crystallization varies depending on the crystallization solvent to be used. Normally, a temperature suitable for crystallization is not lower than −50° C. and not higher than the boiling point of the crystallization solvent. In the present specification, a temperature suitable for crystallization is a temperature during a crystallization step including a temperature at a crystallization initiation time and a temperature at a crystallization termination time. An exemplified crystallization method is a method for heating a crystallization solution and cooling the same to thereby precipitate crystals, which will be described later in detail. In such an exemplified crystallization method, it is recommendable to keep the temperature of the crystallization solution during heating and the temperature of the sufficiently cooled crystallization solution at the crystallization termination time within the above range.

If the crystallization temperature falls below the lower limit of the above range, it gives rise to various disadvantages in the aspect of production cost. Contrary to this, if the crystallization temperature exceeds the upper limit of the above range, evaporation of the solvent used in the crystallization is exceedingly active, which may likely to cause an undesirable change of the concentration of the solvent during crystallization. As a preferred embodiment, there is proposed an approach of using a solvent having the boiling point of 100° C. or higher to carry out crystallization under the condition from −50 to 100° C. Employing such a condition is advantageous in temperature control during crystallization.

Mono(hydroxyethyl) ethers of dihydroxybenzene are difficult to be dissolved at normal temperature depending on the solvent to be used in crystallization, and sometimes turn to a suspended solution (slurry). In such a case, crystallization is carried out after heating the slurry to a solution. An appropriate temperature for heating the slurry is selected from the aforementioned crystallization temperature range.

Although it is a general practice to carry out crystallization under normal pressure, it is also preferable to carry out crystallization under pressurization if a low-boihng-point solvent is used, for example.

There is proposed a crystallization method, wherein, after preparing a solution in which mono(hydroxyethyl) ethers of dihydroxybenzene as a raw material are completely dissolved in a solvent, one of the following methods is carried out: (I) gradually lowering the temperature of the solution; (II) gradually volatizing the solvent; (III) gradually adding the solution to a poor solvent of mono(hydroxyethyl) ethers of dihydroxybenzene; and (IV) gradually pressurizing the solution. It is preferable to vary the state of the crystallization solution while stirring the crystallization system in a crystallization vessel in order to precipitate crystals of mono(hydroxyethyl) ethers of dihydroxybenzene.

Among the above proposed crystallization methods, preferred is a method for gradually lowering the temperature of the solution after mono(hydroxyethyl) ethers of dihydroxybenzene as a raw material are completely dissolved in the solvent. In such a case, the temperature for completely dissolving the material ranges, preferably, from 80 to 100° C. Keeping the temperature at an initial stage of crystallization within the above range facilitates cooling following the crystallization. Further, it is preferable to keep the cooling rate of cooling the crystallization solution obtained by heating at 40° C./hour or less, more preferably 30° C./hour or less, and furthermore preferably 20° C./hour or less. If the cooling rate exceeds the upper limit, it is likely that an exceedingly fast cooling rate may give rise to insufficient purification, thereby resulting in generation of excessively fine crystals of mono(hydroxyethyl) ethers of dihydroxybenzene, thus lowering of the filtration rate in taking out crystals from the crystallization solvent.

As mentioned above, crystals of mono(hydroxyethyl) ethers of dihydroxybenzene are precipitated by changing the state of the crystallization solution such as lowering the temperature of the crystallization solution. Alternatively, it is preferable to charge crystals of mono(hydroxyethyl) ethers of dihydroxybenzene into the crystallization solution to accelerate precipitation of crystals in a case that desirable precipitation of crystals is not obtainable even after a relatively large degree of the change of the state of the crystallization solution is attained.

Crystals of mono(hydroxyethyl) ethers of dihydroxybenzene are unavoidably precipitated in the crystallization solvent by the aforementioned operation. However, as far as the concentration of the crystallization solution prior to crystallization is kept in the aforementioned range, avoided is a phenomenon that the crystallization solvent (slurry) containing crystals generated as a result of crystallization is turned into a viscous paste, which may cause difficulty in stirring the slurry. Further, separation of crystals from the slurry is facilitated.

After the crystals are sufficiently precipitated, a separation step is carried out to take out the crystals from the slurry. Various known separation methods are feasible, e.g., filtration such as filtration under reduced pressure, and pressure filtration, as well as centrifugal separation. The conditions for separation are not specifically limited. The filter fabric and the filter used in filtration are not specifically limited as far as these filter members have a filtration capability of sufficiently filtering the crystals.

After removal of the crystals, the resulting crystallization product is dried by a dryer or its equivalent to yield purified mono(hydroxyethyl) ethers of dihydroxybenzene.

The second production process is effective in suppressing oxidation of unreacted multivalent phenols (generation of quinones) in mono(hydroxyethyl) ethers of dihydroxybenzene in the purification step, namely, in the aromatic ethers, as well as sufficiently removing quinones, if such quinones are generated, in the purification step. The aromatic ethers attain high purity with less coloration. Furthermore, the second production process is advantageous in remarkably reducing the amount of energy required in the purification step as compared with the conventional method adopting a distillation method.

The crystallization-purification step in the second production process is a step of purifying aromatic ethers. Generally, raw material aromatic ethers for preparing a crystallization solution is the one extracted from a reaction solution obtained by synthesis of aromatic ethers according to a known technique. This crystallization technique is also applicable as a crystallization method used in taking out aromatic ethers from the reaction solution obtained by synthesis of aromatic ethers. In view of this, it is recommended to adopt the crystallization-purification technique in the crystallization step following the reaction step in the first production process.

The thus extracted aromatic ethers are purified by applying the crystallization-purification technique used in the second production process in extracting aromatic ethers from the reaction solution after synthesis of aromatic ethers. This means that the aforementioned embodiment is embraced in the invention directed to the second production process.

In the above case, if the solubility parameter of the solvent used in the reaction solution lies within the range from 7.5 to 12.5, it is possible to directly carry out crystallization with respect to the reaction solution without implementing any preprocess. As an alternative, it is possible to carry out crystallization with respect to the reaction solution after regulating the concentration of the solution by feeding a solvent having a solubility parameter ranging from 7.5 to 12.5 or by removing a part of the solvent by distillation or the like. On the other hand, if the solubility parameter of the solvent used in the reaction solution is below 7.5 or above 12.5, it is possible to carry out crystallization by admixing the other solvent in such an amount as to attain the solubility parameter ranging from 7.5 to 12.5 or by substituting a solvent having a solubility parameter ranging from 7.5 to 12.5 for the reaction solvent.

It is recommendable to make the solvent used in the reaction step and the solvent used in the crystallization step identical to each other in the first production process. In view of this, it is desirable to use the solvent having a solubility parameter ranging from 7.5 to 12.5 as a reaction solvent.

The aromatic ethers producible by the first production process and the second production process are usable solely or in combination with other ingredient(s) depending on the purpose of use. Further, the shape and state of the aromatic ethers are not specifically limited, and various forms such as solid (e.g. powder, flakes, and granules), slurry, and solution (e.g. organic solvent solution) are applicable.

Exemplified forms of transportation and storage of the aromatic ethers include the ones in which a diluent, a stabilizer or its equivalent is added to the crude product after the reaction is completed, as well as to the purified product. For instance, it is preferable to render the phenolic hydroxyl group to a light-blocked state by substituting a gaseous phase by inert gas (normally at an oxygen concentration of 0.01 vol % or less, preferably 0.001 vol % or less) in light of preventing oxidation of the phenolic hydroxyl group. Further, it is preferable to use a radical scavenger (with the content of phosphorous acid or diester phosphite from 10 to 100 ppm by mass, for example) as a coexistent agent. It is recommendable to keep the aromatic ethers in a slightly acidic condition (e.g. pH=6 to 7) in light of preventing deterioration of color. For instance, organic acids including aliphatic carboxylic acids (such as formic acid, oxalic acid, citric acid, tartaric acid, glycolic acid, lactic acid, succinic acid, malic acid, and glyceric acid, preferably such as lactic acid and succinic acid in light of being low volatile). The added amount of these organic acids preferably ranges from 1 to 1,000 ppm by mass, more preferably from 5 to 1,000 ppm by mass. The timing of adding these additives (diluent and stabilizer) is not specifically limited. The additives may be added at any time during the reaction step, the crystallization step, and the final-product producing step.

Embodiments

In the following, this invention is illustrated in detail with Examples, which, however, do not limit the invention. Adequate modification is allowable as far as it does not depart from the object of this invention described above or below, and every such modification is intended to be embraced in the technical scope of this invention. It should be noted that throughout the following examples, the unit “ppm” is based in terms of mass.

### CATALYST SYNTHESIZING EXAMPLE

In the following, described is a process for preparing an anion exchange resin A used as a catalyst in the examples of this invention. First, into a separable flask of 1 liter, 350 ml of toluene, and 50 ml of liquid paraffin were charged with addition of 0.07 g of sorbitan palmitate and 0.21 g of ethyl cellulose. The mixture was dissolved to yield a disperse medium. Mixed and dissolved were 41.8 g of diallyldimethyl ammonium chloride aqueous solution having concentration of 65% by mass, 8.3 g of N,N,N′,N′-tetraallyldipiperidyl propanium dichloride (TADPPC, cross-linking agent), and 5.4 g of water to yield a monomer solution. Further, prepared was a solution in which 0.32 g of 2,2′-azobis(2-amidinopropane)dihydrochloride (polymerization initiator, V-50 manufactured by Wako Pure Chemical Industries, Ltd.), and 3.5 g of water were mixed. The solution was added to the monomer solution. TADPPC, a cross-linking agent, is a tetraallylyzed compound obtained by adding allylchloride to 1,3-di(4-piperidyl)propane.

Next, the monomer solution was added to the disperse medium while being stirred, and the admixture was reacted at 55° C. for 4 hours, 60° C. for 16 hours, and then in a temperature range from 92 to 95° C. for 6 hours. After the reaction, particles in the mixture were separated by filtration, and the obtained particles were washed with 600 ml of toluene once, and with 800 ml of methanol thrice. After the washing, the particles were dried in vacuo, and 36.2 g of dried particles were obtained as an anion exchange resin A (Cl-type).

An anion exchange resin A (OH-type) was prepared by the following procedures. The resin A (Cl-type) was swollen in water and charged into a chromato-column. Into the chromato-column, 2N NaOH solution 20 times as much as the resin A in volume, ion exchange water 20 times as much as the resin A in volume, methanol 10 times as much as the resin A in volume were successively passed at a passing rate of SV=2. Thereafter, methanol was removed by vacuum drying, and the anion exchange resin A (OH-type) was obtained.

Experiment 1<Production of Aromatic Ethers by Reaction of Raw Material Phenols with Oxirane Compound Substantially with No Residual of Hydroxyl Group in the Raw Material Phenols>

### EXAMPLE 1

Reaction of adding ethylene oxide (EO) to phenol (monovalent phenol) was carried out according to the following procedures. Into an autoclave of 500 ml equipped with a gas feeding pipe and a stirrer, charged were 90 g of phenol, with addition of 239 g of ethyleneglycol monomethyl ether (δ=11.4) as a solvent, and 15.5 g of anion exchange resin A (Cl-type dried material) as a catalyst, and the autoclave was sealably closed. Subsequently, dissolved oxygen in the solution was removed by deaeration, and a gaseous phase in the autoclave was substituted by nitrogen, and the interior of the autoclave was pressurized to 1 kg/cm²·G. Next, the inner temperature of the autoclave was heated to 100° C., and 48 g of EO was added to the autoclave through the gas feeding pipe for a time duration of 30 minutes. Then, the inner temperature of the autoclave was kept in a range from 90 to 100° C., and thus the reaction was carried out for 6.5 hours. After the reaction, the anion exchange resin A was separated from the reaction solution by filtration.

The reaction solution was analyzed by gas chromatography (GC). The GC analyzing conditions are as follows. GC analysis on all the examples in the present specification was carried out in compliance with the following conditions. GC-15A (manufactured by Shimadzu Corporation) was used as a GC analyzer, DB-1 (Φ: 32 mm, length: 30 m) manufactured by Agilent Technologies (J & W) was used as a column, and helium was used as a carrier. The temperature conditions were such that: the temperature of the column was kept at 70° C. for 5 minutes after charging the reaction solution sample, raised at a heating rate of 15° C. per minute until 300° C., and then retained at 300° C. thereafter. The composition of the sample is shown in terms of area ratio of the corresponding peak shown in the obtained GC chart.

A result of GC analysis reveals that the content of the raw material phenol in the reaction solution was 70 ppm, the content of phenol-1EO adduct (β-phenoxyethanol) was 98.6%, and the content of phenol-2EO adduct was 1.4%. Phenol-2EO adduct is ether obtained by reaction of two EO molecules with a hydroxyl group in phenol.

### EXAMPLE 2

Reaction of adding EO to phenol was carried out in the same manner as in Example 1 except that no solvent was used, and the reaction was implemented in the following condition. Specifically, into an autoclave, charged were 225.8 g of phenol, and 10.0 g of anion exchange resin A (Cl-type dried material) with feeding of hog of EO at 100° C. for 2 hours. Then, after the mixture in the autoclave was aged at 100° C. for 7 hours, the anion exchange resin A was separated from the reaction solution.

Analysis on the composition of the reaction solution according to GC reveals that the content of the raw material phenol in the reaction solution was 90 ppm, the content of β-phenoxyethanol was 95%, and the content of phenol-2EO adduct was 4.9%.

### EXAMPLE 3

Reaction of adding EO to bisphenol S (BPS) as a multivalent phenol was carried out in the same manner as in Example 1 except the following. Specifically, into an autoclave, charged was 100 g of BPS, with addition of 200 g of ethyleneglycol monomethyl ether (δ=11.4) as a solvent, and 13.6 g of anion exchange resin A (Cl-type dried material), and the autoclave was sealably closed. Next, the inner temperature of the autoclave was heated to 100° C., and 44 g of EO was added to the autoclave for a time duration of 30 minutes. Then, the mixture in the autoclave was aged at 100° C. for 5.5 hours. After the aging, the anion exchange resin A was separated from the reaction solution by filtration. Cooling the reaction solution resulted in precipitation of a white solid.

The reaction solution was analyzed according to liquid chromatography (LC) after adding dimethylformamide and uniformly dissolving the mixture. LC analyzing conditions are as follows. Combination of a pump (L-7100) and a UV detector (L-7450H) manufactured by Hitachi Ltd. was used as a LC analyzer. Inertsil ODS (Φ: 4.6 mm, length: 25 cm) manufactured by GL Sciences Inc. was used as a column. 0.1% by mass of a mixed solution of phosphoric acid and methanol (40:60 in volume ratio) was used as a carrier. LC analysis was carried out at a column temperature of 40° C., and a fluid rate of 1 ml/min. The composition of the reaction solution sample is shown in terms of area ratio of the corresponding peak shown in the obtained LC chart.

A result of LC analysis reveals that all the raw material BPS was converted (conversion is 100%), and the content of BPS-2EO adduct was 95% and the content of BPS-3EO adduct was 4.1%. No BPS-1EO adduct was detected. BPS-1EO adduct is ether obtained by reaction of either one of two hydroxyl groups in BPS with one EO molecule. BPS-2EO adduct is ether obtained by reaction of both of two hydroxyl groups in BPS with two EO molecules. BSP-3EO addcut is ether in which one of hydroxyl groups in BSP is reacted with one EO molecule, whereas the other one of the hydroxyl groups in BSP is reacted with two EO molecules.

### EXAMPLE 4

Reaction of adding propyleneoxide (PO) to phenol was carried out. Into a glass pressure-tight vessel of 50 ml, charged were 10.0 g of phenol, and 7.4 g of PO, with addition of 0.8 g of anion exchange resin A (Cl-type dried material) as a catalyst. Subsequently, after the gaseous phase in the vessel was substituted by nitrogen, and the vessel was sealably closed, the vessel was heated in an oil bath of 100° C. accompanied by concussion. After the reaction was carried out for 12 hours, the anion exchange resin A was separated from the reaction solution by filtration.

A result of GC analysis on the composition of the reaction solution reveals that the content of the raw material phenol was 10 ppm, the content of phenol-1PO adduct was 94.4%, and the content of phenol-2PO adduct was 5.3%, respectively. Phenol-1PO adduct is ether obtained by reaction of a hydroxyl group in phenol with one PO molecule. Phenol-2PO adduct is ether obtained by reaction of a hydroxyl group in phenol with two PO molecules.

### EXAMPLE 5

Reaction of adding propylene oxide (PO) to bisphenol A (BPA) was carried out according to the following procedures. Into a glass pressure-tight vessel of 50 ml, charged was 5.0 g of BPA and 2.8 g of PO, with addition of 10.0 g of ethyleneglycol monomethyl ether (δ=11.4) as a solvent, and 0.5 g of anion exchange resin A(OH-type) as a catalyst. Subsequently, after the gaseous phase in the vessel was substituted by nitrogen, and the vessel was sealably closed, the vessel was heated in an oil bath of 100° C. accompanied by concussion. After the reaction was carried out for 6 hours, the anion exchange resin A was separated from the reaction solution by filtration.

A result of GC analysis on the composition of the reaction solution reveals that the content of BPA-1PO adduct was 0.1%, the content of BPA-2PO adduct was 99.0%, and the content of BPA-3PO addcut was 0.8%, respectively. BPA-1PO adduct is ether obtained by reaction of either one of two hydroxyl groups in BPA with one PO molecule. BPA-2PO adduct is ether obtained by reaction of both of two hydroxyl groups in BPA with two PO molecules. BPA-3PO adduct is ether in which one of two hydroxyl groups in BPA is reacted with one PO molecule, whereas the other one of the two hydroxyl groups in BPS is reacted with two PO molecules.

### EXAMPLE 6

Addition reaction of EO to BPA was carried out in the same manner as in Example 1 except for the following. Specifically, into an autoclave, charged was 100 g of BPA, with addition of 200 g of ethyleneglycol monomethyl ether (δ=11.4) as a solvent, and 10.0 g of anion exchange resin A (Cl-type dried material), and the autoclave was sealably closed. Next, into the autoclave, 44 g of EO was added, and the admixture in the autoclave was aged at 100° C. for 7 hours. After the aging, the anion exchange resin A was separated from the reaction solution by filtration.

A result of GC analysis on the composition of the reaction solution reveals that the content of BPA-1EO adduct (BPA-1EO) was 0.4%, the content of BPA-2EO adduct (BPA-2EO) was 97.7%, and the content of BPA-3EO adduct (BPA-3EO) was 1.9%, respectively. No raw material BPA was detected. It should be noted that BPA-1EO adduct (BPA-1EO), BPA-2EO adduct (BPA-2EO), and BPA-3EO adduct (BPA-3EO) respectively correspond to BPA-1PO addcut, BPA-2PO addcut, and BPA-3PO adduct in Example 5, wherein PO is replaced with EO.

### EXAMPLE 7

When the reaction solution obtained in Example 6 was let stand at room temperature, a white solid was started to precipitate. After allowing the reaction solution to stand for 3 hours while cooling the reaction solution to −20° C., the solid was separated by filtration, and dried.

A result of GC analysis on the composition of the solid reveals that the content of BPA-1EO was 0.1%, the content of BPA-2EO was 98.3%, and the content of BPA-3EO was 1.6%, respectively. The yield of BPA-2EO was 80%. It should be noted that the yield is a value relative to a theoretical yield of the reaction product added with oxirane compound which is calculated based on the quantity of the raw material oxirane compound. Hereinafter, the yield is calculated in the same manner.

### EXAMPLE 8

An experiment on reuse of the catalyst was carried out in the same manner as in Example 4 with use of the anion exchange resin A which was used and recovered in Example 4 except that the added amount of EO was 39 g, and the aging time was 9 hours. A result of GC analysis on the resultant reaction solution reveals that the content of BPA-1EO was 0.3%, the content of BPA-2EO was 97.1%, and the content of BPA-3EO was 2.6%, respectively. No BPA was detected. The result on the experiment reveals that the anion exchange resin as a catalyst is reusable.

### EXAMPLE 9

Reaction was carried out in the similar manner as in Example 4 except that 200 g of methanol (δ=14.5) was used as a solvent, and the aging time was 9 hours. A result of GC analysis on the composition of the reaction solution reveals that the content of BPA-1EO was 0.5%, the content of BPA-2EO was 97.7%, and the content of BPA-3EO was 1.9%, respectively. No BPA was detected.

Next, after the reaction solution was allowed to stand at 25° C. for 3 hours, the precipitated solid was recovered and dried. A result of GC analysis on the composition of the solid reveals that the content of BPA-1EO was 0.1%, the content of BPA-2EO was 98.9%, and the content of BPA-3EO was 1.0%, respectively. The yield of BPA-2EO was 70%.

### EXAMPLE 10

Reaction of adding EO to bisphenol fluorene (BPF) was carried out in the similar manner as in Example 1 except for the following. Specifically, into an autoclave, charged were 100 g of BPF, 200 g of ethyleneglycol monomethyl ether (δ=11.4), and 10.0 g of anion exchange resin A (Cl-type dried material), with addition of 31 g of EO. Next, the mixture was reacted in the autoclave at 100° C. for 12 hours. After the reaction, the anion exchange resin A was separated from the reaction solution.

A result of GC analysis on the composition of the reaction solution reveals that the content of BPF-2EO was 97.1%, and the content of BPF-3EO was 2.7%, respectively. No BPF and BPF-1EO were detected.

### EXAMPLE 11

After letting the reaction solution obtained in Example 10 stand at 25° C., a white solid was precipitated. The solid was recovered and dried. A result of GC analysis on the composition of the reaction solution reveals that the content of BPF-2EO was 98.0%, and the content of BPF-3EO was 2.0%, respectively. The yield of BPF-2EO was 60%.

### EXAMPLE 12

Reaction of adding EO to biscresol fluorene (BCF) as a multivalent phenol was carried out in the similar manner as in Example 1 except for the following. Specifically, into an autoclave, charged was 100 g of BCF, with addition of 200 g of ethyleneglycol monomethyl ether (δ=11.4), and 10.0 g of anion exchange resin A (Cl-type dried material), and the autoclave was sealably closed. Next, 29.1 g of EO was added to the mixture, and the mixture was aged in the autoclave at 100° C. for 10 hours. After the aging, the anion exchange resin A was separated from the reaction solution by filtration.

The composition of the reaction solution was analyzed according to LC. The LC analyzing conditions were the same as in Example 3 except for the use of 0.1% by mass of the mixed solution of phosphoric acid and methanol (35:65 in volume ratio). A result of LC analysis on the composition of the reaction solution reveals that all the raw material BCF was converted (inversion rate is 100%), and the content of BCF-2EO adduct was 96.3%, and the content of BCF-3EO adduct was 3.4%, respectively. No BCF-LEO adduct was detected. BCF-1EO adduct is ether obtained by reaction of either one of two hydroxyl groups in BCF with one EO molecule. BCF-2EO adduct is ether obtained by reaction of both of two hydroxyl groups in BCF with two EO molecules. BCF-3EO adduct is ether in which one of two hydroxyl groups in BCF is reacted with one EO molecule, whereas the other one of the two hydroxyl groups in BCF is reacted with two EO molecules.

### EXAMPLE 13

Crystallization-purification of the reaction solution obtained in Example 12 was carried out. Into a separable flask equipped with a stirrer, a cooling device, and a thermometer, charged was a solution for crystallization, which is the reaction solution obtained in Example 12. The flask was heated in an oil bath at 95° C. Next, the solution was cooled from 95° C. to 40° C. at a cooling rate of 5° C./hour while stirring the solution at 200 rpm. As a result of stirring, the solution was turned into a slurry having fluidity in its entirety. Then, the slurry was retained at 40° C. for 1 hour. Thus, crystallization was terminated. The slurry was subjected to filtration, and the filtrate was washed with 40 g of ethylene glycol monomethyl ether at normal temperature. Then, the filtrate was depressurized and dried at 60° C., thereby yielding the purified product. As is the case of Example 12, a result of LC analysis on the composition of the purified product reveals that the content of BCF-2EO adduct was 99.2%, the content of BCF-3EO adduct was 0.8%, respectively, and the yield of BCF-2EO adduct was 60%.

Experiment 2<Production of Aromatic Ethers Having a Phenolic Hydroxyl Group>

### EXAMPLE 2-1

Reaction of adding EO to catechol was carried out according to the following procedures. Into an autoclave of 500 ml equipped with a gas feeding pipe and a stirrer, charged was 100 g of catechol, with addition of 200 g of ethyleneglycol monomethyl ether (δ=11.4) as a solvent, and 14 g of anion exchange resin A (Cl-type dried material) as a catalyst, and the autoclave was sealably closed. Subsequently, after dissolved oxygen in the solution was removed by deaeration, and the gaseous phase in the autoclave was substituted by nitrogen, the autoclave was pressurized. Next, the inner temperature of the autoclave was heated to 100° C., and 44 g of EO was added to the autoclave through the gas feeding pipe for a time duration of 30 minutes. Then, the inner temperature of the autoclave was kept at 100° C., and the mixture was aged for 3 hours. After the reaction was completed, the anion exchange resin A was separated from the reaction solution by filtration.

An analysis on the composition of the reaction solution was carried out by GC. The analysis reveals that 88% of the raw material catechol was reacted, and the ratio of a target compound [catechol-1EO adduct, namely, β-(2-hydroxyphenoxy)ethanol] to the reaction product was 82%. Hereinafter, the ratio is called as “reaction selectivity”.

### EXAMPLE 2-2

Reaction was carried out in the similar manner as in Example 2-1 except that toluene was used as a solvent. A result of reaction is shown in Table 1. After the reaction was completed, the reaction solution was subjected to pressure filtration while keeping the temperature at 100° C. Then, the resin A was separated from the reaction solution. The reaction solution was allowed to stand at room temperature, and allowed to cool. Then, a white solid was precipitated. A GC analysis (according to analytical curve) on the composition of the solid reveals that the content of catechol (CC) was 0.6% by mass, the content of CC-1EO adduct was 96.8% by mass, and the content of CC-2EO adduct was 2.6% by mass, respectively, with yield of the white solid of 85 g. It should be noted that CC-2EO adduct is ether obtained by reaction of both of two hydroxyl groups in catechol with two EO molecules.

The content of sodium (metal) in the solid was measured according to an inductively coupled plasma spectrometry. An analyzer “SPS4000” manufactured by Seiko Instruments Inc. was used as a spectrometer in measuring the content of metal. A measurement result reveals that the content of sodium was less than 1 ppm relative to the total content of the solid.

The content of halogen element in the solid was measured according to an X-ray fluorescence spectrometry. An analyzer “PW2404” manufactured by Philips Japan, Ltd. was used as a spectrometer in measuring the content of halogen. In the analysis, the qualitative analysis program installed in the spectrometer was used, and quantitative determination was carried out by comparing the solid with standard specimens of halogen elements (fluorine, chlorine, bromine, and iodine). A measurement result reveals that no halogen element was verified in the solid only with the content thereof of less than 100 ppm.

### EXAMPLES 2-3, 2-4, and 2-5

Experiments were carried out in the same manner as in Example 2-1 by varying the reaction conditions. The reaction conditions and reaction results are shown in Table 1. Note that EQ was added for 3 hours in Examples 2-4 and 2-5.

### EXAMPLE 2-6

The anion exchange resin A used in Example 2-1 was separated from the reaction solution and recovered by filtration under reduced pressure. Next, the recovered anion exchange resin A was washed with 300 ml of methanol, and dried in vacuo. Then, the reaction was carried out in the similar manner as in Example 1 by using the recovered anion exchange resin A as a catalyst, and an experiment on reuse of the catalyst was implemented. As is obvious from Table 1, the anion exchange resin as a catalyst does not show a remarkable deterioration in catalytic activity, and thus is reusable.

### EXAMPLE 2-7

Reaction of adding PO to catechol was carried out according to the following procedures. Specifically, into a glass pressure-tight vessel of 50 ml, charged were 5.0 g of catechol, and 3.2 g of PO, with addition of 10.0 g of ethyleneglycol monomethyl ether (δ=11.4) as a solvent, and 0.8 g of anion exchange resin A (Cl-type dried material) as a catalyst. Subsequently, after the gaseous phase in the vessel was substituted by nitrogen, and the vessel was sealably closed, the vessel was heated in an oil bath of 90° C. accompanied by concussion. After the reaction was completed, the anion exchange resin A was separated from the reaction solution by filtration.

An analysis on the composition of the reaction solution was carried out by GC. As a result of carrying out the reaction for 12 hours, 89% of the raw material catechol was reacted, and the reaction selectivity of a target compound [catechol-1EO adduct, namely, α-methyl-β(2-hydroxyphenoxy)ethanol and β-methyl-β-(2-hydroxyphenoxy)ethanol] to the reaction product was 84%.

### EXAMPLES 2-8 through 2-14

Experiments were carried out in the same manner as in Example 2-7 by varying the reaction conditions. The reaction conditions and reaction results are shown in Table 1. The anion exchange resin B in Example 2-10 is diaion TSA1200 (heat resistive anion exchange resin manufactured by Mitsubishi Chemical Corporation, used in the form of dehydrated material of chlorine ion).

TABLE 1

| | | | | | | | Conversion | Reaction |
|---|---|---|---|---|---|---|---|---|
| | | | | Solvent | | | of | selectivity |
| | Multivalent | Oxirane | | Kind, | | Reaction | multivalent | of target |
| | phenols | compound | Catalyst | Amount | δ | condition | phenols | compound |
| Ex. | CC | EO | resin A | EGMME | 11.4 | 100° C. | 88% | 82% |
| 2-1 | 100 g | 44 g | 14 g | 200 g | | 3.5 hr | | |
| Ex. | CC | EO | resin A | toluene | 8.9 | 100° C. | 94% | 83% |
| 2-2 | 100 g | 44 g | 14 g | 200 g | | 3.5 hr | | |
| Ex. | CC | EO | resin A | toluene | 8.9 | 100° C. | 77% | 91% |
| 2-3 | 100 g | 44 g | 14 g | 200 g | | 1.5 hr | | |
| Ex. | HQ | EO | resin A | EGMME | 11.4 | 100° C. | 73% | 69% |
| 2-4 | 100 g | 48 g | 14 g | 200 g | | 4.5 hr | | |
| Ex. | HQ | EO | resin A | EGMME | 9.7 | 100° C. | 77% | 73% |
| 2-5 | 100 g | 48 g | 14 g | 67 g + toluene | | 6 hr | | |
| | | | | 133 g | | | | |
| Ex. | CC | EO | recovered | EGMME | 11.4 | 100° C. | 86% | 83% |
| 2-6 | 100 g | 44 g | resin A | 200 g | | 3.5 hr | | |
| Ex. | CC | PO | resin A | EGMME | 11.4 | 90° C. | 89% | 84% |
| 2-7 | 5.0 g | 3.2 g | 0.8 g | 10.0 g | | 12 hr | | |
| Ex. | CC | PO | resin A | toluene | 8.9 | 90° C. | 94% | 87% |
| 2-8 | 5.0 g | 3.2 g | 0.8 g | 10.0 g | | 12 hr | | |
| Ex. | CC | PO | resin A | MIBK | 8.4 | 90° C. | 80% | 89% |
| 2-9 | 5.0 g | 3.2 g | 0.8 g | 10.0 g | | 8 hr | | |
| Ex. | CC | PO | resin B | toluene | 8.9 | 90° C. | 96% | 84% |
| 2-10 | 5.0 g | 3.2 g | 0.9 g | 10.0 g | | 12 hr | | |
| Ex. | HQ | PO | resin A | EGMME | 11.4 | 90° C. | 84% | 73% |
| 2-11 | 5.0 g | 3.2 g | 0.8 g | 10.0 g | | 8 hr | | |
| Ex. | HQ | PO | resin A | toluene | 8.9 | 90° C. | 93% | 74% |
| 2-12 | 5.0 g | 3.2 g | 0.8 g | 10.0 g | | 12 hr | | |
| Ex. | RC | PO | resin A | EGMME | 11.4 | 100° C. | 82% | 70% |
| 2-13 | 5.0 g | 3.4 g | 0.8 g | 10.0 g | | 4 hr | | |
| Ex. | RC | PO | resin A | toluene | 8.9 | 100° C. | 79% | 76% |
| 2-14 | 5.0 g | 3.4 g | 0.8 g | 10.0 g | | 4 hr | | |

The symbols in Table 1 indicate the following:
CC: catechol
HQ: hydroquinone
RC: resorcin
EO: ethylene oxide
PO: propylene oxide
resin A: anion exchange resin A
resin B: anion exchange resin B
TMAC: tetramethyl ammonium chloride
EGMME: ethyleneglycol monomethyl ether
MIBK: methyl isobutyl ketone
δ: solubility parameter

In “reaction selectivity of target compound”, the target compound is an aromatic ether having a phenolic hydroxyl group (β-phenoxyethanol), and is a compound in which one molecular oxirane compound is added to a multivalent phenol in each example. The reaction selectivity is expressed in terms of area ratio relative to the total area corresponding to all the reaction products in the obtained GC analysis chart. Time (reaction time) in the reaction conditions is defined such that the start time of adding EO or PO is 0 hour.

### REFERENCE EXAMPLE 1

Reaction of adding PO to catechol was carried out according to the following procedures. Into a glass pressure-tight vessel of 50 ml, charged were 5.00 g of catechol, and 3.16 g of PO, with addition of 10.0 g of toluene (δ=8.9) as a solvent, and 88 mg of tetramethyl ammonium chloride as a catalyst. Then, the gaseous phase in the vessel was substituted by nitrogen, and the vessel was sealably closed. The vessel was heated in an oil bath of 100° C. accompanied by concussion for 4 hours. The reaction solution was analyzed according to GC, and calculated were inversion rate of catechol, selectivity of mono(hydroxypropyl) ether (mono-type) and bis(hydroxypropyl) ether (bis-type), and yield of the mono-type compound, which are respectively defined as below.

Conversion (%) of catechol=100×(number of moles of consumed catechol/number of moles of supplied catechol)

Selectivity (%) of mono-type (bis-type) compound=100×[number of moles of generated mono-type (bis-type) compound/number of moles of consumed catechol]

Yield (%) of mono-type compound=(conversion of catechol)×(selectivity of mono-type compound)÷100

The results of calculation are shown in Table 2.

### REFERENCE EXAMPLES 2, 3

Reaction of adding PO to catechol was carried out in the same manner as in Reference Example 1 except that 258 mg of tetrabutyl ammonium bromide (Reference Example 2) or 437 mg of tetraoctyl ammonium bromide (Reference Example 3) was used as a catalyst. Results on GC analysis are shown in Table 2.

### REFERENCE EXAMPLES 4 through 6

Reaction of adding PO to catechol was carried out in the same manner as in Reference Example 1 except that 437 mg of tetraoctyl ammonium bromide was used as a catalyst, and butyl acetate (δ=8.5) (Reference Example 4), ethyleneglycol monomethyl ether (δ=11.4) (Reference Example 5) or dimethylformamide (δ=12.1) (Reference Example 6) was used as a solvent. Results on GC analysis are shown in Table 2.

### COMPARATIVE EXAMPLE

Reaction of adding propylene oxide to catechol was carried out in the same manner as in Reference Example 1 except that 437 mg of tetraoctyl ammonium bromide was used as a catalyst, and water (δ=23.4) was used as a solvent. A result of GC analysis is shown in Table 2.

TABLE 2

| | | | | Analysis result on reaction solution | | | |
|---|---|---|---|---|---|---|---|
| | | | | | Selectivity of | | Yield of |
| | | Solvent | | Conversion of | mono-type | Selectivity of | mono-type |
| | Catalyst | Kind | δ | catechol (%) | (%) | bis-type (%) | (%) |
| R. Ex. 1 | Me₄NCl | toluene | 8.9 | 81.3 | 84.9 | 15.1 | 69.0 |
| R. Ex. 2 | Bu₄NBr | toluene | 8.9 | 88.2 | 84.2 | 15.8 | 74.2 |
| R. Ex. 3 | Oc₄NBr | toluene | 8.9 | 85.4 | 85.1 | 14.9 | 72.6 |
| R. Ex. 4 | Oc₄NBr | butyl acetate | 8.5 | 84.3 | 85.7 | 14.3 | 72.2 |
| R. Ex. 5 | Oc₄NBr | EGMME | 11.4 | 84.8 | 83.7 | 16.3 | 70.9 |
| R. Ex. 6 | Oc₄NBr | DMF | 12.1 | 74.2 | 81.2 | 18.8 | 60.2 |
| C. Ex. | Oc₄NBr | water | 23.4 | 63.0 | 88.9 | 11.1 | 56.0 |

The symbols in Table 2 indicate the following:

Me₄NCl: tetramethyl ammonium chloride

Bu₄NBr: tetrabutyl ammonium bromide

Oc₄NBr: tetraoctyl ammonium bromide

EGMME: ethyleneglycol monomethyl ether

DMF: dimethylformamide

δ: solubility parameter

Mono-type: mono(hydroxypropyl) ether of catechol

Bis-type: bis(hydroxypropyl) ether of catechol

Experiment 3<Production of Aromatic Ethers by Crystallization-Purification with use of a Crystallization Solvent Having a Specific Solubility Parameter>

“Part” and “%” in Experiment 3 are units in terms of mass unless otherwise specified.

[Synthesis Example] Synthesis of Mono(hydroxyethyl) Ether of Catechol

### SYNTHESIS EXAMPLE 1

Into a simplified autoclave of 1,000 ml equipped with a stirrer, a pressure gauge, a feed pipe, and a liquid drainage pipe mounted with a metal gauze, charged were 200.1 parts of catechol, 403.1 parts of toluene (δ=8.9), and 27.4 parts of anion exchange resin B. After the reaction system was substituted by nitrogen, the reaction system was heated to 100° C. Thereafter, 92 parts of EO was charged into the autoclave for a time duration of 5 hours while keeping the reaction system at 100° C. After charging of EO was completed, the reaction system was aged for 4 hours at 100° C., and then the reaction was terminated. The reaction solution was drained through the liquid drainage pipe mounted with a metal gauze while keeping the reaction solution at 100° C. Thus, a transparent and colorless reaction solution was obtained. A result of GC analysis on the reaction solution reveals that the reaction solution contains unreacted catechol, mono(hydroxyethyl) ether of catechol, bis(hydroxylethyl) ether of catechol, bis(hydroxyethoxyethyl) ether of catechol, and ether in which one hydroxyl group in catechol is converted to hydroxyethoxyethoxy group, while the other one hydroxyl group in catechol is converted to hydroxyethoxy group [(hydroxyethoxyethyl)(hydroxyethyl) ether of catechol]. The contents of the respective compounds to the total content of all the components in the reaction solution (=100%) are shown in Table 3.

### SYNTHESIS EXAMPLE 2

Into a simplified autoclave of 1,000 ml equipped with a stirrer, a pressure gauge, a feed pipe, and a liquid drainage pipe mounted with a metal gauze, charged were 200.0 parts of catechol, 400.0 parts of ethyleneglycol monomethyl ether (δ=11.4), and 18.4 parts of anion exchange resin B. After the reaction system was substituted by nitrogen, the reaction system was heated to 100° C. Thereafter, 88 parts of EO was charged into the autoclave for a time duration of 5 hours while keeping the reaction system at 100° C. After charging of EO was completed, the reaction system was aged for 4 hours at 100° C., and then the reaction was terminated. The reaction solution was drained through the liquid drainage pipe mounted with a metal gauze while keeping the reaction solution at 100° C. Thus, a transparent and colorless reaction solution was obtained. A result of GC analysis on the reaction solution is shown in Table 3.

### SYNTHESIS EXAMPLE 3

Into a simplified autoclave of 1,000 ml equipped with a stirrer, a pressure gauge, a feed pipe, and a liquid drainage pipe mounted with a metal gauze, charged were 200.0 parts of catechol, 400.0 parts of ethyleneglycol monomethyl ether (δ=11.4), and 2.0 parts of potassium hydroxide. After the reaction system was substituted by nitrogen, the reaction system was heated to 100° C. Thereafter, 92 parts of EO was charged into the autoclave for a time duration of 4 hours while keeping the reaction system at 100° C. After charging of EO was completed, the reaction system was aged for 3 hours at 100° C., and then the reaction was terminated. The reaction solution was drained through the liquid drainage pipe mounted with a metal gauze. A result on GC analysis of the reaction solution is shown in Table 3.

### EXAMPLE 3-1

100 parts of the reaction solution obtained in Synthesis Example 1 was heated to 80° C., and filtered under pressure filtration with use of a filter. The residual amount of the catalyst in the filtrate was less than 0.1% relative to the total amount of all the reaction products except the solvent. The filtrate was charged into a separable flask of 500 ml equipped with a stirrer, a cooling device, and a thermometer with addition of 110 parts of toluene (δ=8.9). Thus, a slurry having a solid content of 20% was obtained. The slurry was heated in an oil bath to 80° C. to dissolve crystals in the slurry. Thereby, the slurry was turned into a solution.

Thereafter, the solution was stirred at 200 rpm by the stirrer, and cooled from 80° C. to 30° C. at a cooling rate of 5° C./hour. As a result of cooling, the solution was turned into a slurry having fluidity in its entirety. Thereafter, the slurry was kept at 30° C. for 1 hour. Thus, crystallization was terminated. The slurry was taken out from the separable flask, and filtered. The slurry was taken out desirably from the separable flask with no substance adhered on the flask wall. The filtrate was washed with 20 parts of toluene at normal temperature. Thereafter, the filtrate was dried by a vacuum dryer, and a purified product was obtained. The result of GC analysis on the purified product is shown in Table 3. The yield of the purified product was 82% to the total amount of all the reaction products obtained in Synthesis Example 1.

### EXAMPLE 3-2

An experiment was carried out in the same manner as in Example 1, and a purified product was obtained except that 320 parts of toluene was added, and the slurry having a solid content of 10% was obtained. A result of GC analysis on the purified product is shown in Table 3. The yield of the purified product was 74% to the total amount of all the reaction products obtained in Synthesis Example 1.

### EXAMPLE 3-3

Into a separable flask of 500 ml equipped with a stirrer, a pressure gauge, a thermometer, and a distiller, charged was 200 parts of the reaction solution obtained in Synthesis Example 2. The reaction solution was heated to 110° C. under reduced pressure of 0.0266 MPa, and 73 parts of ethyleneglycol monomethyl eter ((δ=11.4) was taken out. The solution was filtered under pressure filtration with use of a filter. The residual amount of the catalyst in the filtrate was less than 0.1% relative to the total amount of all the reaction products except the solvent. The filtrate was charged into a separable flask of 500 ml equipped with a stirrer, a cooling device, and a thermometer with addition of 170 parts of butyl acetate (δ=8.5). Thus, a slurry having a solid content of 28% was obtained. The solubility parameter 6 mix of the mixed solvent consisting of methoxyethanol in the concentrated reaction solution and the added butyl acetate was 9.0 by implementing calculation according to the equation (10). The slurry was heated to 80° C. in an oil bath to dissolve crystals in the slurry. Thereby, the slurry was turned into a solution.

Thereafter, the solution was stirred at 200 rpm by the stirrer, and cooled from 80° C. to 30° C. at a cooling rate of 5° C./hour. As a result of cooling, the solution was turned into a slurry having fluidity in its entirety. Thereafter, the slurry was kept at 30° C. for 1 hour. Thus, crystallization was terminated. The slurry was taken out from the separable flask, and filtered. The slurry was taken out desirably from the separable flask with no substance adhered on the flask wall. The filtrate was washed with 40 parts of butyl acetate at normal temperature. Thereafter, the filtrate was dried by a vacuum dryer, and a purified product was obtained. The result of GC analysis on the purified product is shown in Table 3. The yield of the purified product was 76% to the total amount of all the reaction products obtained in Synthesis Example 2.

### EXAMPLE 3-4

200 parts of the reaction solution obtained in Synthesis Example 3 was depressurized by an aspirator with use of an evaporator while being heated to 60° C., thereby distilling off ethyleneglycol monomethyl ether. Into a separable flask of 500 ml equipped with a stirrer, a cooling device, and a thermometer, charged was 80 parts of the solid obtained by distillation with addition of 320 parts of toluene (δ=8.9). Thus, a slurry having a solid concentration of 20% was obtained. The slurry was heated to 80° C. in an oil bath to dissolve crystals in the slurry. Thereby, the slurry was turned into a solution.

Thereafter, the solution was stirred at 200 rpm by the stirrer, and cooled from 80° C. to 30° C. at a cooling rate of 5° C./hour. As a result of cooling, the solution was turned into a slurry having fluidity in its entirety. Thereafter, the slurry was kept at 30° C. for 1 hour. Thus, crystallization was terminated. The slurry was taken out from the separable flask, and filtered. The filtrate was washed with 40 parts of toluene at normal temperature. Thereafter, the filtrate was dried by a vacuum dryer, and a purified product was obtained. A result of GC analysis on the purified product is shown in Table 3. The yield of the purified product was 66% to the total amount of all the reaction products obtained in Synthesis Example 3.

### COMPARATIVE EXAMPLE 3-1

Into a separable flask of 500 ml equipped with a stirrer, a cooling device, and a thermometer, charged were 100 parts of the reaction solution obtained in Synthesis Example 2 and 110 parts of ethanol (δ=12.7) to make a slurry having a solid content of 20%. The slurry was heated to 80° C. in an oil bath to dissolve crystal in the slurry. Thereby, the slurry was turned into a solution. Thereafter, the solution was stirred at 200 rpm by the stirrer, and cooled from 80° C. to 5° C. at a cooling rate of 5° C./hour. As a result of cooling, no crystal was precipitated, thus resulting in failure of crystallization.

### COMPARATIVE EXAMPLE 3-2

Into a separable flask of 500 ml equipped with a stirrer, a cooling device, and a thermometer, charged were 100 parts of the reaction solution obtained in Synthesis Example 1 and 110 parts of n-hexane (δ=7.3) to make a slurry having a solid content of 20%. The slurry was heated to 80° C. in an oil bath. However, no crystal in the slurry has been dissolved.

### EXAMPLE 3-5

Into a separable flask of 500 ml equipped with a stirrer, a cooling device, and a thermometer, charged was 100 parts of the reaction solution obtained in Synthesis Example 1 to make a slurry having a solid content of 42%. The slurry was heated to 80° C. in an oil bath to dissolve crystals precipitated in the slurry. Thereby, the slurry was turned into a solution. Thereafter, the solution was stirred at 200 rpm by the stirrer, and cooled from 80° C. to 30° C. at a cooling rate of 5° C./hour. As a result of cooling, the precipitated crystals were turned into agglomerate, thus failing to obtain a slurry in a stable state. The slurry was taken out and filtered. The filtrate was washed with 20 parts of toluene, and then dried by a vacuum dryer. Thus, a purified product was obtained. A result of GC analysis on the purified product is shown in Table 3. The yield of the purified product was 85% to the total amount of all the reaction products obtained in Synthesis Example 1.

TABLE 3

| | Solubility | | | | | |
|---|---|---|---|---|---|---|
| | parameter | | | | | |
| | of | Content (%) | | | | |
| | crystallization solvent | a | b | c | d | e |
| Synthesis Ex. 1 | — | 3.4 | 80.4 | 0.9 | 15.0 | 0.3 |
| Synthesis Ex. 2 | — | 3.6 | 74.1 | 1.0 | 21.0 | 0.3 |
| Synthesis Ex. 3 | — | 3.2 | 67.0 | 1.1 | 28.3 | 0.4 |
| Ex. 3-1 | 8.9 | 0.1 | 99.5 | 0.0 | 0.4 | 0.0 |
| Ex. 3-2 | 8.9 | 0.0 | 99.4 | 0.0 | 0.6 | 0.0 |
| Ex. 3-3 | 9.0 | 0.1 | 96.9 | 0.1 | 2.9 | 0.0 |
| Ex. 3-4 | 8.9 | 0.1 | 95.9 | 0.1 | 3.9 | 0.0 |
| Ex. 3-5 | 8.9 | 0.6 | 95.4 | 0.2 | 3.7 | 0.1 |

a: catechol
b: catechol, mono(hydroxyethyl) ether
c: catechol, mono(hydroxyethoxyethyl) ether
d: catechol, bis(hydroxyethyl) ether
e: catechol, bis(hydroxyethoxyethyl)(hydroxyethyl) ether

This application is based on Japanese patent application No. 2002-217744, No. 2002-247284, and No. 2003-100529 filed on Jul. 26, 2002, Aug. 27, 2002, and Apr. 3, 2003 respectively, the contents of which are hereby incorporated by references.

As this invention may be embodied in several forms without departing from the spirit of essential characteristics thereof, the present embodiment is therefore illustrative an not restrictive, since the scope of the invention is defined by the appended claims rather than by the description preceding them, and all changes that fall within metes and bounds of the claims, or equivalence of such metes and bounds are therefore intended to embraced by the claims.

What is claimed is:

1. A process for producing aromatic ethers comprising a step of reacting phenols with an oxirane compound with use of an anion exchange resin as a catalyst.

2. The process according to claim 1, wherein the phenols include multivalent phenols, and the aromatic ethers producible by the reaction contain a phenolic hydroxyl group and an alcoholic hydroxyl group.

3. The process according to claim 1, wherein the reaction of the phenols with the oxirane compound is carried out in the presence of a solvent having a solubility parameter ranging from 7.0 to 20.0.

4. The process according to claim 1, wherein the phenols include phenol or cresol.

5. The process according to claim 1, wherein the phenols include catechols, resorcinols, or hydroquinones.

6. The process according to claim 5, wherein the phenols include catechol, resorcinol, or hydroquinone.

7. The process according to claim 1, wherein the phenols include bisphenols.

8. The process according to claim 7, wherein the phenols include bisphenol A, bisphenol S, bisphenol fluorene, or biscresol fluorene.

9. The process according to claim 1, wherein the oxirane compound includes ethylene oxide, propylene oxide, isobutylene oxide, or 2,3-butylene oxide.

10. The process according to claim 1, further comprises a crystallization step following the reaction step, wherein a solvent used in the crystallization step is identical to a solvent in the reaction step in kind, and at least a partial amount of the solvent in the crystallization is used in the reaction step in using the solvent in the reaction step.

11. A process for producing aromatic ethers having an alcoholic hydroxyl group comprising a crystallization-purification step of using a solvent having a solubility parameter ranging from 7.5 to 12.5 for purification by crystallization.

12. Aromatic ethers having an alcoholic hydroxyl group, wherein the content of a metal in the aromatic ethers is less than 100 ppm by mass, and the content of a halogen element in the aromatic ethers is less than 100 ppm by mass.
